SEQUENCE LISTING (2 sequences)

SEQ ID NO: 1 - DNA, 24 bp, Artificial Sequence
  Primer
agaaggagaaggaaagggaaaggg

SEQ ID NO: 2 - DNA, 24 bp, Artificial Sequence
  Primer
caccaacccaaaccaacccaaacc

(19) United States
(12) Patent Application Publication
(10) Pub. No.: US 2010/0304445 A1
(43) Pub. Date: Dec. 2, 2010

(54) NUCLEIC ACID PACKAGING SYSTEM

(76) Inventors: Waclaw Szybalski, Madison, WI (US); Frederick Blattner, Madison, WI (US); David Frisch, Madison, WI (US); Douglas Wieczorek, Madison, WI (US)

Correspondence Address: POLSINELLI SHUGHART PC, 700 W. 47TH STREET, SUITE 1000, KANSAS CITY, MO, 64112-1802, US

(21) Appl. No.: 12/664,899

(22) PCT Filed: Jun. 16, 2008
(86) PCT No.: PCT/US08/67160

§ 371 Date: Jul. 12, 2010

Related U.S. Application Data
(60) Provisional application No. 60/944,494, filed on Jun. 16, 2007.

(51) Int. Cl. C12N 15/64; C12P 19/34

(52) U.S. Cl. 435/91.4; 435/91.5

(57) ABSTRACT

The present invention relates to cloning target nucleic acids using phage packaging mechanisms. Packaging initiation sites may be introduced into the target DNA. Components of a phage packaging system may be combined with the target DNA to package the DNA into phage capsids. The packaged DNA may be used to create a library of target nucleic acids, or it may be sequenced.

### BACKGROUND

1. Field of the Invention

The present invention relates generally to the field of molecular biology. More specifically, the present invention concerns the cloning of nucleic acid molecules and the production of nucleic acid libraries.

2. Description of Related Art

Genomics has become a central and cohesive discipline of biomedical research. Genome sequencing projects generate a steady stream of ever-larger and more complex genomic data sets that have transformed the study of virtually all life processes. Advances in genetics and comparative genomics allow disease to be analyzed and comprehended at an unprecedented level of molecular detail.

Genomic research requires the availability of high quality, large insert genomic libraries. High quality genomic libraries require DNA fragments with a narrow size range. Size selection of DNA fragments is typically performed using pulsed field gel electrophoresis (PFGE), which has several limitations. PFGE is extremely time consuming and irreproducible. Moreover, the yield of fragment DNA from PFGE is extremely poor.

High quality genomic libraries also require the entire genome to be represented with minimal bias. DNA fragments for genomic libraries are often produced using restriction enzymes to partially digest the genome, which has several limitations. Partial digestion of the genome leads to bias against regions of the genome where the distance between restriction sites is less than or greater than the applied size-selection limits. As an alternative to the cloning bias associated with partial digestion, DNA fragments can be produced by randomly shearing the genome. However, cloning efficiencies are significantly reduced using randomly sheared DNA.

What the art needs are high quality genomic libraries that are able to faithfully represent an entire genome. The genomic libraries should be produced efficiently and with high yield. The genomic libraries should also be easily propagated and analyzed. The present invention satisfies these needs.

### SUMMARY

The present invention is related to the use of packaging systems for cloning and sequencing a nucleic acid and for cloning and sequencing a plurality of nucleic acids. The packaging system may be, for example, a phage-based packaging mechanism. The nucleic acids may be packaged into capsids, which may be a bacteriophage capsids. A cloned nucleic acid may be used, for example, in the production of a nucleic acid library.

Provided herein is a method of sequencing the nucleic acid, which may comprise providing a capsid comprising the nucleic, isolating the nucleic acid and sequencing the nucleic acid. Also provided herein is a method of sequencing a plurality of nucleic acids, which may comprise providing a plurality of capsids, each comprising a nucleic acid; isolating the nucleic acids; and sequencing the nucleic acids.

The sequencing may comprise (a) delivering a nucleic acid into an aqueous microreactor in a water-in-oil emulsion such that the aqueous microreactor comprises a single copy of the nucleic acid, a single bead capable of binding to the nucleic acid, and amplification reaction solution containing reagents necessary to perform nucleic acid amplification; (b) amplifying the nucleic acid in the microreactor to form amplified copies of the nucleic acid and binding the amplified copies to the bead in the microreactor; (c) delivering the bead to an array of at least 10,000 reaction chambers on a planar surface, wherein a plurality of the reaction chambers comprise no more than a single nucleic acid bound bead; and (d) performing a sequencing reaction simultaneously on a plurality of the reaction chambers.

The sequencing may also comprise (a) delivering a plurality of nucleic acids into an aqueous microreactor in a water-in-oil emulsion such that a plurality of aqueous microreactors comprise a single copy of a nucleic acid, a single bead capable of binding to the nucleic acid, and amplification reaction solution containing reagents necessary to perform nucleic acid amplification; (b) amplifying the nucleic acids in the microreactor to form amplified copies of the nucleic acids and binding the amplified copies to the bead in the microreactor; (c) delivering the beads to an array of at least 10,000 reaction chambers on a planar surface, wherein a plurality of the reaction chambers comprise no more than a single nucleic acid bound bead; and

(d) performing a sequencing reaction simultaneously on a plurality of the reaction chambers.

The bead may bind at least 10,000 amplified copies. Amplifying the nucleic acid may be accomplished by using the polymerase chain reaction. The sequencing reaction may be a pyrophosphate-based sequencing reaction. The sequencing reaction may comprise (a) annealing an effective amount of a sequencing prier to the amplified copies of the nucleic acid an extending the sequencing primer with a polymerase and a predetermined nucleotide triphosphate to yield a sequencing product and if the predetermined nucleotide triphosphate is incorporated onto a 3′ end of the sequencing primer, a sequencing reaction byproduct; and (b) identifying the sequencing reaction byproduct, thereby determining the sequence of the nucleic acid in a reaction chamber.

Isolating a nucleic acid or plurality of nucleic acids may comprise (a) ligating a first adaptor end and a second adaptor end to the at least one nucleic acid, wherein the first adaptor end comprises an oligonucleotide sequence Y and ligates to the 5′ end of the at least one nucleic acid, the second adaptor end comprises an oligonucleotide sequence Z and ligates to the 3′ end of the at least one nucleic acid, and first adaptor carries a means for immobilizing the at least one nucleic acid to a solid support at the 5′ end; (b) mixing the at least one nucleic acid of step (a), in the presence of the solid support, with one or more colony primers X, each of which can hybridize to the oligonucleotide sequence Z and carries a means for immobilizing the colony primer to the solid support at the 5′ end, whereby the 5′ ends of both the at least one nucleic acid and the colony primers are immobilized to the solid support; wherein said 5′ ends of both the at least one nucleic acid and the colony primers are immobilized to said solid support such that they cannot be removed by washing with water or aqueous buffer under DNA-denaturing conditions; and (c) performing one or more nucleic acid amplification reactions on the immobilized at least one nucleic acid, so that nucleic acid colonies are generated. The colony primers may be degenerate.

The oligonucleotide sequence Z may be complementary to oligonucleotide sequence Y and colony primer X may be of the same sequence as oligonucleotide sequence Y. Two different colony primers X may be mixed with the at least one nucleic acid when ligating the adaptor ends to the nucleic acid, and the oligonucleotide sequence Z may hybridize to one of the colony primers X and the oligonucleotide Y may be the same as the sequence of one of the colony primers X.

The at least one nucleic acid may be sequenced in one or more of the nucleic acid colonies. The sequencing may involve incorporating and detection of labeled nucleotides, and may also comprise visualizing the nucleic acid colonies. Visualizing may involve the use of a labeled or unlabeled nucleic acid probe.

The means for immobilizing the at least one nucleic acid and the colony primers to the solid support may comprise means for immobilizing the at least one nucleic acid and the colony primer covalently to the support. The means for immobilizing the at least one nucleic acid and the colony primers covalently to the solid support may be a chemically modified group. The means for immobilizing the at least one nucleic acid and the colony primers to the solid support may comprise means for immobilizing the at least one nucleic acid and the colony primers covalently to the support. The chemically modifiable functional group may be an amino group.

The solid support to which said 5′ ends of both the at least one nucleic acid and the colony primers are immobilized may be selected from the group consisting of latex beads, dextran beads, polystyrene and polypropylene surfaces, polyacrylamide gel, gold surfaces, glass surfaces, and silicon wafers. The density of the nucleic acid colonies on the solid support may be 10,000/mm² to 100,000/mm². The density of the colony primers λ attached to the solid support may be at least 1 fmol/mm². The density of the at least one nucleic acid may be 10,000/mm² to 100,000/mm². The 5′ ends of both the at least one nucleic acid and the colony primers may be immobilized to said solid support via covalent attachment.

Also provided herein is a method for cloning the nucleic acid. A packaging initiation site (PIS) may be introduced into a target nucleic acid, which establishes the upstream end of a nucleic acid to be cloned from the target nucleic acid. The PIS may be a phage packaging initiation site including, but not limited to, λ, P1, P7 or T4. The target nucleic acid may be any form of nucleic acid including, but not limited to, a vector, chromosomal DNA, or an entire genome. The use of an entire genome as the target nucleic acid may allow the production of genomic libraries.

The PIS may be randomly introduced into the target nucleic acid, which may provide for increased coverage of the genome. The PIS may be introduced into the target nucleic acid in any manner including, but not limited to, transposition and ligation. The PIS may be introduced by transposition of a first nucleic acid. The first nucleic acid may comprise transposable ends including, but not limited to, Tn7, Tn5, Tn/O, Mu and Mariner.

Packaging may then be initiated at the PIS, which leads to the cloning of nucleic acid downstream from the PIS extending into the target nucleic acid. Packaging may occur in vitro or in vivo. The cloned nucleic acid may be packaged in a capsid, which may allow clones of substantially uniform lengths.

The first nucleic acid may also comprise a vector element. The vector element may be downstream of the PIS and may also be present in the cloned nucleic acid. The vector element may be an origin of replication including, but not limited to, a low copy origin of replication and a high copy origin of replication. The vector element may also be a low copy origin of replication together with a high copy origin of replication. The high copy origin of replication may be responsive to a replication-inducing agent. The first nucleic acid may comprise a nucleic acid encoding the replication-inducing agent. The replication-inducing agent may be TrfA.

### BRIEF DESCRIPTION OF THE DRAWINGS

FIG. 1 demonstrates a methodology for transposon-mediated cloning using λdoc particles. Step 1: An in vitro transposition reaction randomly inserts the PAC/oriV containing transposon at multiple sites throughout the genomic DNA (long lines) via transposable ends (outward pointing arrows); Step 2: DNA is packaged in vitro starting at the cos site and continuing until the head is full. Protruding DNA is then digested with Sau3A, while DNA within the head is protected; Step 3: Phage heads are then purified and lysed resulting in fragments of uniform length and the general composition of cos-PAC/oriV (thick arrow) fused to genomic DNA with a Sau3A overhang. Oligos with Sau3A and cos overhangs are then ligated to the fragments; Step 4: Fragments are re-packaged and used to infect a host such as DH10B where the incoming DNA circularizes through the cos ends. Clones are isolated as Cam^{R} colonies.

FIG. 2 shows an additional scheme for cloning via in vitro transposition and phage packaging.

FIG. 3 shows a linker comprising two nucleic acids (SEQ ID NOS: 1 and 2) to add a cos site to a DNA fragment with a Sau3A overhang.

FIG. 4 demonstrates the procedure for in vivo recombination based on use of transposon-mediated cloning. Step 1: As in FIG. 1, an in vitro transposition reaction randomly inserts a transposon at multiple site throughout the genomic DNA (long lines) via transposable ends (outward pointing arrows). However, in this case the transposon contains cos-attB-Kan^{R} with no origin of replication; Step 2: DNA is packaged in vitro starting at the cos site and continuing until the head is full. Protruding DNA is then digested with Sau3A, while DNA within the head is protected; Step 3: Phage heads are then purified and lysed resulting in fragments of uniform length and the general composition of cos-PACIoriV (thick arrow) fused to genomic DNA with a Sau3A overhang. Oligos with Sau3A and cos overhangs are then ligated to the fragments; Step 4: Fragments are re-packaged and used to infect a host such as DH10B that contains a new pPAC/oriV/attP vector and has been induced to express Int. Following cyclization via the cos ends results in a Cam^{R} conferring plasmid.

FIG. 5 shows a transposable/packagable vector. PvuII cleavage of the vector results in a linear fragment flanked by Tn5 mosaic ends (ME) allowing transposition in vitro. pac and cos sites then permit the transposed product to be packaged in vitro.

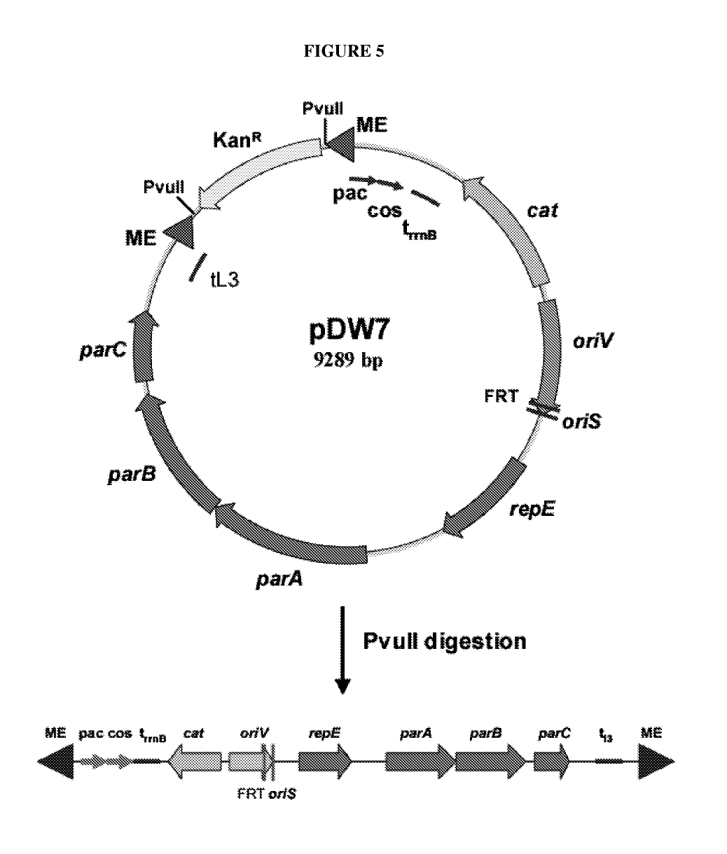

FIG. 6 shows a strategy for generating a genomic DNA library using a λ phage-based packaging system followed by affinity purification of phage capsids and isolation of packaged DNA.

FIG. 7 shows a strategy for generating a genomic DNA library using a P22HT “headful” packaging system.

FIG. 8 shows a strategy for cloning and characterizing the sequence of ends of viral capsid-packaged DNA.

FIG. 9 shows how cloning a class III restriction enzyme site and reaching primer pair binding sites into phage-packaged DNA can be used to characterize the sequence of the ends of the DNA.

FIG. 10 shows how the ends of phage-packaged DNA can be PCR-amplified after cloning a reaching primer pair insert into the DNA.

FIG. 11 shows how the ends of phage-packaged DNA can be sequenced after cloning a reaching primer pair insert into the DNA.

### DETAILED DESCRIPTION

The inventors have developed a phage packaging system that can be used to isolate and package target DNA with more faithful representation. Depending on the phage packaging system used, the system generates DNA fragments of a particular, more uniform size. In addition, the target DNA is more ligatable compared to methods requiring shearing or restriction digest steps to reduce the target DNA to a manageable and clonable size. Accordingly, the efficiency of obtaining the target DNA is higher compared to other methods. Following packaging of the target DNA, the DNA may be used for numerous purposes, such as generating a library, it may be extracted, it may be amplified, or the DNA may be sequenced, such as by cloning and sequencing the ends of the packaged DNA fragments.

DNA fragments may be generated by introducing packaging initiation sites (PIS) into the target DNA. The PIS establishes the upstream end of a nucleic acid to be cloned from a target nucleic acid. Recognition of the PIS by the packaging system may lead to initiation of packaging at the PIS, which leads to cloning of the nucleic acid downstream from the PIS extending into the target nucleic acid. A library of nucleic acids may be produced by cloning a plurality of nucleic acids from a target nucleic acid. A genomic library may be produced by cloning a plurality of nucleic acids from a genome.

1. Packaging Systems

A nucleic acid may be cloned from a target nucleic acid by initiating packaging at a packaging initiation site (PIS). The PIS may be recognized by a terminase, which may site-specifically or randomly nick the target nucleic acid, and which may insert the nucleic acid into a capsid. The cloned nucleic acid may thus be packaged in a capsid. The terminase may comprise two proteins, which may have ATPase activity that enables the packaging system to function.

The capsid may be capable of packaging a nucleic acid that may be 5-160 kb in size. A plurality of capsids may each be capable of packaging a nucleic acid, and the nucleic acids packaged by the capsids may be of approximately the same size. Such a form of size selection is a major improvement over the inefficiencies associated with PFGE electrophoresis. Moreover, the infectious properties of capsids may allow introduction of the packaged nucleic acid at higher rates than electroporation in subsequent manipulations.'

The packaging system used may be eukaryotic or prokaryotic. For example, the system may be selected from a bacteriophage listed in Table 1.

TABLE 1

| | | | Nucleic |
|---|---|---|---|
| Phage Family or Group | Example | Capsid (nm) | Acid (MW) |
| Caudovirales | T4, λ, T7 | 67 | 79 |
| Range | | 30-160 | 17-498 |
| Microviridae | φX174 | 27 | 4.4-6.1 |
| Corticoviridae | PM2 | 60 | 9.0 |
| Tectiviridae | PRD1 | 63 | 15 |
| Leviviridae | MS2 | 23 | 3.5-4.3 |
| Cystoviridae | φ6 | 75-80 | 13.4 |
| Inoviridae, Inovirus, | fd | 760-1950 × 7 | 5.8-7.3 |
| Plectrovirus | | | |
| | L51 | 85-250 × 7 | 4.4-8.3 |
| Lipothrixviridae | TTV1 | 400-2400 × 20-40 | 16-42 |
| Rudiviridae | SIRV1 | 780-900 × 23 | 33-36 |
| Plasmaviridae | L2 | 80 | 11.7 |
| Fuselloviridae | SSV1 | 85 × 55 | 15 |

The packaging system may also be selected from a poxvirus, herpesvirus, adenovirus, lentivirus, or Epstein-Barr virus.

a. Packaging Initiation Site

The PIS may be a packaging initiation site. A representative example of a packaging initiation site includes, but is not limited to, a bacteriophage λ, P1, P7, T4, T7, Φ29, or P22 packaging initiation site. The PIS may be a pac or a cos site. The PIS may also be a eukaryotic virus PIS, such as a long terminal repeat.

The PIS may be introduced into the target nucleic acid by introducing into the target nucleic acid a first nucleic acid comprising the PIS. The first nucleic acid may also comprise a vector element. The first nucleic acid may also comprise a transposable element. The transposable element may comprise a pair of transposable ends flanking a nucleic acid segment comprising the PIS. The transposable element may comprise transposable ends from systems including, but not limited to, Tn7, Tn5, Tn/O, Minos, Mu and Mariner. The nucleic acid segment may also comprise a vector element.

The first nucleic acid may be introduced into the target nucleic acid in either a random or non-random manner. The first nucleic acid comprising the PIS may also be introduced into the target nucleic acid by any of a number of standard molecular biology methodologies including, but not limited to, mutagenesis. The first nucleic acid comprising the PIS may be introduced into the target nucleic acid by transposition, either in vitro or in vivo. The first nucleic acid may be in any form that allows transposition of the transposable element into the target DNA. The first nucleic acid may be in the form of a linear or circular DNA, which may be supercoiled or relaxed. The first nucleic acid may be part of a plasmid. The first nucleic acid may also be a replicable genetic package including, but not limited to, a bacteriophage.

Transposase may be used in vitro or in vivo to catalyze the transposition of the first nucleic acid into the target nucleic acid. Transposition may occur randomly, the degree of which may differ depending on the specific transposon system.

Transposition may occur in vitro using a commercially available transposition system, for example. Transposition may also occur in vitro using methods disclosed in U.S. Pat. Nos. 5,965,443, 5,948,622. or 5,925,545, the contents of which are incorporated herein by reference. For in vitro transposition, the target nucleic acid may be any form of nucleic acid including, but not limited to, chromosomal DNA, digested DNA, sheared DNA, size-specific DNA, and an entire genome.

Transposition may also occur in vivo by introducing the first nucleic acid into a host cell that expresses transposase. The first nucleic acid may be introduced into the host cell by methods including, but not limited to, electroporation and infection. For in vivo transposition, the target nucleic acid may be any form of nucleic acid, including a replicon. The replicon may be a vector or a chromosome.

The first nucleic acid comprising the PIS may also be introduced into the target nucleic acid by ligating the first nucleic acid to the target nucleic acid. The ligated product may be linear or circular. The target nucleic acid may be any form of nucleic acid including, but not limited to, the forms of nucleic acid discussed above for in vitro transposition. The target nucleic acid may be generated by methods including, but not limited to, random shearing and restriction digestion. The first nucleic acid and the target nucleic acid may have compatible ends. Alternatively, a linker may be used.

The target nucleic may be digested, which may be partial, with a first restriction enzyme such as HindIII. The target nucleic acid may also be digested with a second restriction enzyme. The first nucleic acid may have an end that is compatible with the digested target nucleic acid. After ligating the first nucleic acid and the target nucleic acid, the resulting target nucleic acid comprising the PIS may be a substrate for phage packaging.

The PIS may be native to the target nucleic acid, or may pre-exist in the target DNA. The PIS may also be introduced into the target nucleic acid by DNA cleavage. The cleavage may be performed by contacting the target nucleic acid with a terminase, which may cut the target nucleic acid at a random location. The terminase may be a phage terminase such as from phage

P22, which may be a high transducing P22. The terminase may comprise polypeptides Gp3 and Gp2 of phage P22.

b. Vector Element

The PIS may be introduced into the target nucleic acid along with a vector element. The vector element may be downstream or upstream of the PIS. A downstream PIS may be included in the cloned nucleic acid. The vector element in the cloned nucleic acid may be used for subsequent manipulation and propagation of the cloned DNA.

(1) Origin of Replication

The vector element may be an origin of replication. The origin of replication may be a low copy origin of replication. A low copy origin of replication may be used to maintain the cloned DNA in a host cell while also minimizing rearrangements. A representative example of a low copy origin of replication is oriS.

The origin of replication may also be a high copy origin of replication. A high copy origin of replication may be used to produce higher quantities of the cloned DNA. A representative example of a low copy origin of replication is oriV.

The vector element may also be a low copy origin of replication together with a high copy origin of replication. The high copy origin of replication may be under the control of an inducible promoter. The cloned nucleic acid may be maintained using the low copy origin of replication, but may be produced in large quantities by inducing the high copy origin of replication. Such a system is described by Hradecna et al. 1998 and Wild et al. 1998, the contents of which are incorporated herein by reference.

(2) Markers

The vector element may also be a selectable or visible marker. Selectable or visible markers may be used to maintain and manipulate the cloned DNA. Representative examples of markers include, but are not limited to, Amp^{R}, Cam^{R}, Kan^{R}, lacZ and GFP.

(3) Integration Site

The vector element may also be an integration site. The integration site may allow the cloned nucleic acid to be integrated into a second nucleic acid comprising a complementary integration site. The second nucleic acid may also comprise a vector element. Use of an integration system has several important advantages. Many sequences that are required for later manipulation of the cloned nucleic acid may be placed on a replicating plasmid vector instead of the first nucleic acid. This may allow more target nucleic acid to be cloned when using capsids to package the cloned nucleic acid.

Examples of integrations sites and complementary integration sites include, but are not limited to, the λ system and the Cre-lox system. When λ phage infects E. coli, it may either develop lytically or establish a lysogen by integrating at a specific point in the bacterial chromosome, attB. Integration is mediated by site-specific recombination between attP on λ and attB resulting in linear insertion of λ into the continuity of the chromosome. attP is about 250 by long while attB is only about 20 bp. The product of the phage int gene together with the host factor, IHF, catalyzes the crossover reaction between attP and attB thereby causing integration.

The first nucleic acid may comprise attB downstream of the PIS and the second nucleic acid is replicable and comprises attP. After initiating packaging, the cloned may be integrated into the replicable second nucleic acid. Using a similar methodology, DNA fragments have been inserted into the Janus vector (Burland et al. 1993), which is incorporated herein by reference.

c. λ Packaging

The packaging system may use a specific site to initiate and terminate packaging. λ is not a “headful” type of phage because in normal phage development the length of DNA per virion is not determined directly by the capacity of the head. Instead λ employs highly specific cos sites to initiate and terminate packaging. These sites are cut by terminase leaving 12 base overhangs (cosL and cosR) at the ends of the packaged DNA. In λ' s natural rolling circle packaging substrate cos sites are spaced closer together than the full packaging limit determined by head size. As a result, cos site spacing, and not head capacity, normally determines the length of virion DNA.

d. Headful Packaging

A “headful” packaging system may also be used. A headful packing system may feed the cloned nucleic acid into the cavity of a phage prohead in a linear processive manner causing the head to expand until it reaches a limit where the DNA inside exerts pressure against the inner wall sufficient to stop progression. This may induce a conformational change in the head, which activates endonucleolytic cleavage of incoming DNA opening the way for attachment of phage tails to make infectious particles. Full heads may contain DNA molecules within a narrow size range. The capacity of the capsids may set a maximum size limitation on the packaged DNA.

Representative headful packaging systems include, but are not limited to, P1, P7, T4, KVP40, P22, Φ29, and T7.

(1) P1

Bacteriophages P1 and P7 have a headful capacity of 110-115 kb. Packaging initiation occurs at the pac site and continues until the head is full and a non-specific terminal cut is made. Commercially available stage 1 P1 packaging extracts, or pacase extract, may cleave the DNA to be packaged at the pac site, analogous to the cos site of λ. It may contain the am10.1 mutation, a nonsense mutation in the P1 gene 10, that is defective for all late phage protein synthesis, including head and tail proteins. The stage 2 extract may contain the phage packaging proteins that encapsidate the DNA into a head and provide for the addition of the tail. It may contain the am9.16 nonsense mutation of gene 9 that is defective for pac-cleavage activity.

Packaging strains may also be constructed for a two-step packaging reaction. For packaging strain construction, a nonsense mutation may be created in one of the major tail genes including, but not limited to, Tub, encoding the major tail tube protein, or gene 22, encoding the major tail sheath protein. This may lead to a pacase-head proficient/tail deficient stage 1 packaging extract. A stage 2 pacase-head deficient/tail proficient strain may be constructed by introducing a nonsense mutation in gene 23, encoding the major head protein of P1. It may also be desirable to substitute the Cam^{r} marker within the P1 genome.

The cre-lox site-specific recombination system may be used to circularize the P1-packaged cloned nucleic acid. The first nucleic acid may include a loxP site downstream of the pac packaging initiation sequence. After transposition and packaging of the cloned nucleic acid, protruding DNA from the P1 head may be trimmed and linkers added as has been proposed for λ, this time containing a second loxP recombination site. Following the addition of tails during the stage 2 packaging reaction, the packaged DNA may be injected into a Cre+host. Following injection into the host cell, the DNA may be circularized by recombination between two phage loxP sites by Cre recombinase. This may allow the circular, cloned nucleic acid to be replicated and stably maintained.

(2) T4

Bacteriophage T4 is one of the largest and well characterized phages, with a genome size of 169 kb Like P1 and P7, T4 packages its DNA in a headful manner, and the components of the packaging mechanisms have been studied in depth. Unlike λ, P1, and P7, packaging of T4 DNA does not initiate at a specific site. In vitro packaging systems for T4 have been developed. The T4 in vitro system can efficiently package foreign DNA and can be adapted to clone large DNA fragments of up to 160 kb in length.

A similar method described above for P1 and P7 in vitro packaging may be employed making use of the cre-lox site-specific recombination system that is used to circularize the T4-packaged clone. One advantage to the T4 system is the identification of a large number of head-length variants, both larger and smaller, in which the amount of DNA packaged is altered accordingly. Using packaging strains in combination with particular head-length mutations may prove beneficial in expanding the potential size range of cloned nucleic acids.

(3) KVP40

KVP40 is a broad host range vibriophage that has been shown through comparative genomics to be T4-related. It is a large, tailed, double stranded DNA phage with a genome size of 245 kb. While little is currently known regarding the genetics and biochemical activities of this phage, the genome sequence and organization of KVP40 shows regions of extensive conservation with T4. One of the largest conserved regions lies in the gene cluster which makes up the virion structural genes, suggesting similarities in their DNA packaging mechanisms as well. This makes KVP40 a candidate for a cloning system using in vitro packaging.

The development of a KVP40 in vitro packaging system would be required for this extension. With the increased knowledge of T4 DNA packaging, whose major components are well characterized, analogous strains may be developed for in vitro packaging extracts of KVP40. Putative terminase, head, and tail-associated genes have already been identified and will certainly provide for potential mutagenic targets for strain construction.

While the host range of KVP40 includes at least 8 Vibrio and 1 Photobacterium species, it does not infect E. coli, a more desirable host. The ompK gene of V. parahaemolyticus encodes the outer membrane protein (OMP) that has been identified as the host receptor for KVP40. Expression of the ompK gene in an E. coli strain, such as JM109, allows infection by KVP40.

(4) P22

Bacteriophage P22 is a podovirus that may infect strains of Salmonella typhimurium. P22 packages as much as around 42 kb of DNA using a headful mechanism that utilizes a terminase comprising a large subunit (Gp2) and a small subunit (Gp3). Terminase may bind to and cleave DNA at a pac or pac-like site and may then be involved in ATP hydrolysis-based translocation of the DNA into a viral procapsid. DNA may be packaged into the procapsid until the procapsid is full. During packaging, the capsid shell may stretch and undergo a conformational change. Following packaging, the terminase may cleave the end of the packaged DNA again and may then be expelled, still attached to the broken, unpackaged DNA. The terminase may then insert into a new procapsid and continue the process of packaging adjacent target nucleic acid fragments in successive cycles.

(a) P22 Mutant

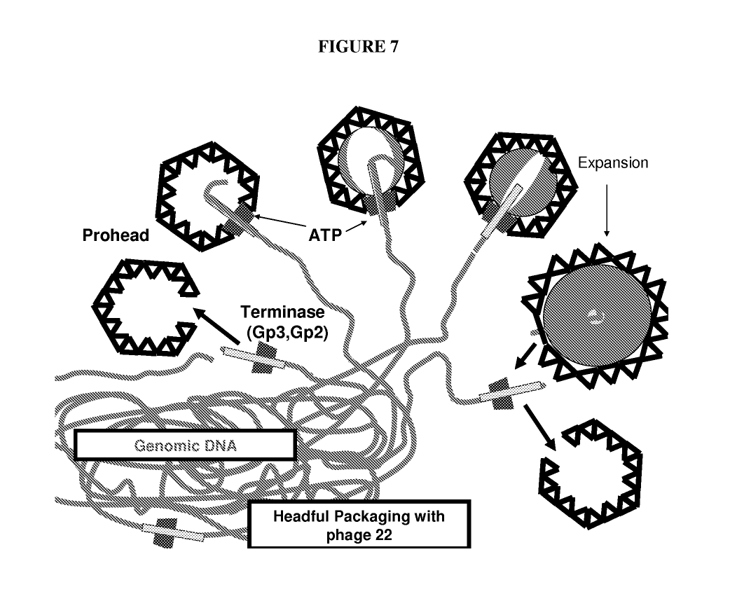

The P22 may be a P22 mutant, such as a high-transducing P22 (P22HT). The P22HT mutation may occur in gene 3, which may result in a mutant Gp3 protein. The P22HT may carry a HT105/1 mutation. Initiation of packaging by the P22HT terminase may have little or no apparent sequence specificity and thus may recognize DNA with lower specificity. The terminase may bind to and cleave DNA at random locations and may then be involved in ATP hydrolysis-based translocation of the DNA into a viral procapsid (FIG. 7). The target nucleic acid packaged by P22HT may be 46 kb.

The P22 mutant may also have increased processivity along a given target nucleic acid. The mutant may be capable of packaging up to ten capsids from a target nucleic acid. The P22 mutant may also be capable of infecting E. coli.

(5) Φ29

Φ29 is a bacteriophage that may infect strains of Bacillus subtilis. The phage may pack as much as around 30 kb of DNA inside a prolate icosahedral capsid. DNA may be translocated into a procapsid by an ATP-dependent motor that comprises a head-tail connector (Gp10), a packaging ATPase (Gp16), and a ring of RNA molecules (pRNA).

(6) λ Packaging and Doc Particles

The nucleic acid to be packaged may contain rarely spaced cos sites. Packaging may start at a cos site, cosL, which is cut leaving a 12 base overhang that is inserted into the head. The cos site may be present at the end of the target nucleic acid by virtue of the first nucleic acid having been ligated to the end of the target nucleic acid. Packaging may proceed unidirectionally until the head is full. If terminase does not find a second cos site to cut, the process may stall with DNA hanging out of the head. The protruding DNA may be removed nonspecifically by a nuclease, such as DNAseI, or a frequently cutting restriction enzyme, such as Sau3A. The position at which the nuclease cuts the protruding DNA may be random.

Phage tails may then be attached producing particles with only one cos site called λdocL. These particles may inject their DNA into E. coli effectively but it may not cyclize efficiently upon entering the cell because they lack cosR, the cohesive end normally found at the right end of the λ molecule. λdocL particles may be used to introduce nucleic acid into a host cell by integration. As an alternative to tail addition, a second round of packaging may be performed. After nuclease digestion and DNA extraction, linkers may be ligated and the subsequent products repackaged, transfected, and clones selected.

The loxP system may be used for in vitro circularization of clones as an alternative to tail addition. A loxP site may be added downstream of the PIS on the first nucleic acid. The cloned nucleic acid may be size-selected by in vitro packaging, extracted, and linkers containing a loxP site ligated. The clones may be circularized by Cre recombinase in vitro and electroporated into a host cell to be stably maintained as a plasmid.

2. Isolating Packaged DNA

After encapsidation, the packaged DNA may be recovered by isolating the capsid. The capsid may be isolated by methods including, but not limited to, isopycnic or velocity centrifugation, or differential sedimentation. The capsid may also be isolated by affinity purification, which may be performed by using an antibody capable of specifically binding to an epitope of a protein that is contained on the surface of an extended capsid. For example, the capsid protein may be a D protein of λ phage.

The anti-capsid protein antibody may be attached to a solid support. The solid support may be a bead, microparticle, column, test strip, cartridge, microtiter plate, microscope slide, or membrane such as nylon, nitrocellulose, or other suitable material. The microparticle may be 0.2 μm-7.0 μm in size. The microparticle may also be haptenated. The microparticle may also be impregnated by at least one or two fluorescent labels. The microparticle may also be capable of forming a ferrofluid or magnetic particle less than about 0.1 μm in size. The microparticle may also be removable by collectable or removable by filtration.

The packaged DNA may be isolated from the capsid, such as by phenol extraction. Prior to extraction, a nuclease such as DNaseI may be used to digest unpackaged DNA or non-target DNA. Phenol may be added to a solution comprising the capsid. After centrifuging this mixture, the aqueous phase may be separated from the organic phase, and chloroform may be added. After centrifuging this mixture, the aqueous phase may be separated from the organic phase. The isolated DNA may be concentrated from the aqueous phase by adding cold ethanol to the aqueous phase, followed by centrifugation. The DNA may be washed one or more times with ethanol, and may be resuspended in water or buffer. The phenol-chloroform extraction may be repeated at least once.

The packaged DNA may also be isolated from the capsid using a DNA-binding column. For example, a QIAGEN lambda procedure (QIAGEN, Valencia, Calif.) may be used. Unpackaged or non-target DNA may be digested with a nuclease. The capsid may be precipitated and then lysed. The isolated DNA may be bound to a column and washed. The DNA may be eluted from the column and precipitated from solution with an alcohol such as ispropanol. The DNA may be resuspended in water or a buffer.

If the capsid is isolated using a capsid protein affinity column, the capsid may be eluted and lysed. The packaged DNA may be phenol extracted or isolated using a column-based system as described above.

The ends of the isolated DNA may be filled-in, which may be done by using a DNA polymerase. An adaptor insert may be ligated to the ends of the DNA, which may result in a circular DNA. The ligation reaction may comprise the adaptor insert at a concentration sufficient for each end of the adaptor insert to ligate to an end of an isolated DNA molecule. The ends of the adaptor insert may not be phosphorylated, which may prevent the adaptor insert from forming a monomer circle. The ligation reaction may be performed in a water-in-oil emulsion. The aqueous droplets of the emulsion may contain approximately one isolated DNA molecule to be circularized.

The adaptor insert may comprise two adaptor primer binding sites, which may have opposite orientations, such as tail-to-tail. The binding sites may be 10-50 or 16-25 base pairs in length. The adaptor primer insert may also comprise priming regions capable of supporting both amplification and nucleotide sequencing. The primers that may bind the sites may have a sequence as set forth in Intl. Pub. No. WO 2007/145612 A1 or U.S. Pat. No. 7,115,400 or 7,323,305, the contents of which are incorporated herein by reference. For example, the reaching primers may be a primer A and a primer B. The adaptor insert may also comprise a short (e.g., 4 nucleotides) “sequencing key” sequence that may be used for well finding on a 454 Sequencing System (454 Life Systems, Branford, Conn.). The adaptor insert may also comprise a separator element, which may have a known sequence. The sequence may comprise a priming site for rolling circle amplification. The sequence may also be used to identify the two ends of the isolated DNA. During subsequent sequencing of the isolated DNA, the sequence of the separator element may indicate that the entire isolated DNA has been sequenced. The adaptor insert may comprise an origin of replication, and may also comprise a marker.

The adaptor insert may also comprise a recognition sequence for a restriction enzyme, such as a blunt-end cutting restriction enzyme, and this sequence may be located in between the two adaptor primer binding sites. Upon ligating the adaptor insert to the isolated DNA, the resulting DNA may be digested with the restriction enzyme capable of cutting the DNA between the adaptor primer binding sites. Adaptor ends may then be ligated to the ends of the resulting linear DNA.

The adaptor insert may also comprise a “reaching” restriction enzyme recognition site. The recognition site may be adjacent to a primer binding site. The adaptor insert may also comprise a reaching restriction enzyme recognition site flanking the two adaptor primer binding sites, and the recognition sites may be oriented in opposite directions away from the restriction enzyme recognition sites. The reaching restriction enzyme may be a class IIS enzyme, which may be Fok I, Alw26 I, Bbv I, Bsr I, Ear I, Hph I, Mme I, Mbo II, SfaN I, or Tth111I. The reaching restriction enzyme may also be a class III restriction enzyme, which may be EcoP15 I, EcoP I, Hinf III, or StyLT I. Upon ligation of the adaptor insert to the isolated DNA, the reaching restriction enzyme may cut DNA a location that is away from the recognition site, such as at a location within the isolated DNA. The adaptor insert may also comprise a specific binding member, which may be biotin.

After ligating the insert to the isolated DNA, the resulting circular DNA may be digested with the reaching restriction enzyme, which may result in a linear DNA fragment, which on each end may comprise a sequence which may correspond to an end of the isolated DNA. The DNA fragment may be circularized, which may be performed by filling-in the ends of the DNA fragment and then performing a ligation.

An adaptor ends comprising an adaptor primer binding site may also be ligated to an end of the isolated DNA. The adaptor end may comprise priming regions capable of supporting both amplification and nucleotide sequencing, and may also comprise a short (e.g., 4 nucleotides) “sequencing key” sequence that may be used for well finding on a 454 Sequencing System (454 Life Systems, Branford, Conn.). The primer capable of binding the site may be primer A or primer B. The adaptor end may be a first adaptor end having oligonucleotide sequence Y. The adaptor end may also be a second adaptor end having oligonucleotide sequence Z. The adaptor end may comprise means for immobilizing the nucleic acid to a solid support.

The adaptor ends may have degenerate two-base single stranded 3′ overhangs. Degenerate may mean that the two overhanging bases may be random (i.e., that they may each be either G, A, T, or C). The adaptor ends may be designed to strongly favor directional ligation of the adaptors to the reaching restriction enzyme-cut ends of the isolated DNA. The adaptor ends may be combined with the isolated DNA ends in a ligation reaction that may contain a large molar excess of adaptor ends (15:1 adaptor end:isolated DNA ratio), which may maximize utilization of the isolated DNA ends and may minimize the potential of forming isolated DNA concatemers. The adaptor ends may not be phosphorylated, which may minimize the formation of adaptor end dimers. Following ligation, the ligation product may be repaired by using a fill-in reaction, such as by using a strand-displacing DNA polymerase. The DNA polymerase may be Bst DNA polymerase (Large Fragment), Φ29 DNA Polymerase, DNA Polymerase I (Klenow Fragment), or Vent® DNA Polymerase.

The adaptor end may be designed as described in U.S. Pat. No. 7,323,305, the contents of which are incorporated herein by reference. The adaptor ends may comprise a phosphorothioate linkage instead of a phosphodiester linkage. The adaptor end may also comprise a specific binding member, which may be biotin. The specific binding member may be added to a first adaptor end, which may be at the 5′ end, and the specific binding member may not be added to a second adaptor end. Following ligation of the first and second adaptor end to the isolated DNA, the isolated DNA may comprise two first adaptor end, one first adaptor end and one second adaptor end, and two second adaptor ends. The adaptor end-ligated isolated DNA may be contacted with a solid substrate bound to a specific binding partner for the specific binding member of the first adaptor end, such as streptavidin. The solid substrate may be a magnetic bead.

Upon contacting the adaptor-end ligated isolated DNA with the solid substrate, only isolated DNA comprising at least one first adaptor end will be bound. Isolated DNA comprising only one first adaptor end will be bound at one end of the isolated DNA, while isolated DNA comprising two first adaptor ends will be bound at two ends. The unbound isolated DNA may be washed away from the solid substrate. The solid substrate may be subjected to low salt (“melt” or denaturing) solution, which may release only isolated DNA comprising only one first adaptor end. Isolated DNA comprising two first adaptor ends will remain bound to the solid substrate. The isolated, single-stranded DNA that is released from the solid substrate may be collected for further use, such as in PCR amplification and sequencing.

3. Propagating the Packaged DNA

After encapsidation, the cloned nucleic acid may also be used to infect an appropriate host cell. In the host cell, the cloned nucleic acid may be circularized and propagated. The cloned nucleic acid may then be isolated using standard methodologies for isolation of DNA. In order to prevent IS contamination of the cloned nucleic acid, the host strain may not comprise an IS element.

If the cloned nucleic acid is not circular, it may be isolated from the capsid and circularized in vitro. Circularization may be performed by methods including, but not limited to, ligating one or more linkers compatible with the ends of the linear DNA or recombination.

The circularized nucleic acid may comprise an ori and a selectable or visible marker. The circularized nucleic acid may be a nucleic acid capable of being propagated in a host cell, such as a bacterium, and may be a bacterial artificial chromosome (BAC). The circularized nucleic acid may be transformed into the host cell.

4. Amplifying the Isolated DNA

The isolated DNA may be amplified, such as by PCR. If the isolated DNA is circularized, it may be linearized by digesting it with a restriction enzyme that is capable of cleaving the DNA at a site located between the adaptor primer binding sites.

a. Emulsion PCR

The isolated DNA comprising adaptor primer binding sites may be PCR amplified, such as by using bead emulsion PCR amplification (emPCR). emPCR may be performed as described in U.S. Pat. No. 7,323,305 and Intl. Pub. No. WO 2007/145612, the contents of which are incorporated herein by reference. The isolated DNA may be single stranded, and may be annealed to an adaptor primer. The adaptor primer may be attached to solid support, which may be a spherical bead. The solid support may comprise a plurality of the bound adaptor primer. The solid support may be suspended in aqueous reaction mixture and then encapsulated in a water-in-oil emulsion. The emulsion may comprise discrete aqueous phase microdroplets, which may be 60-100 μm in diameter, and may be enclosed by a thermostable oil phase. Each microdroplet may contain, amplification reaction solution (i.e., the reagents necessary for nucleic acid amplification). The amplification may comprise a PCR reaction mix (polymerase, salts, dNTPs) and a pair of adaptor primers (primer A and primer B). A subset of the microdroplet population may also contain the DNA bead comprising the DNA template. This subset of microdroplet may be the basis for the amplification. The microcapsules that are not within this subset may have no template DNA and may not participate in amplification. The amplification technique may be PCR, and the PCR primers are present in a 8:1 or 16:1 ratio (i.e., 8 or 16 of one primer to 1 of the second primer) to perform asymmetric PCR.

The isolated DNA may be annealed to an oligonucleotide (primer B) which may be immobilized to a bead. During thermocycling, the bond between the single stranded DNA template and the immobilized B primer on the bead may be broken, which may release the template into the surrounding microencapsulated solution. The amplification solution may contain addition solution phase primer A and primer B. Solution phase B primers may readily bind to the complementary b′ region of the template as binding kinetics are more rapid for solution phase primers than for immobilized primers. In early phase PCR, both A and B strands may amplify equally well.

By midphase amplification (i.e., between cycles 10 and 30) the B primers may be depleted, which may halt exponential amplification. The reaction may then enter asymmetric amplification and the amplicon population may become dominated by A strands. In late phase amplification, after 30 to 40 cycles, asymmetric amplification may increase the concentration of A strands in solution. Excess A strands begin to anneal to bead immobilized B primers. Thermostable polymerases then utilize the A strand as a template to synthesize an immobilized, bead bound B strand of the amplicon.

In final phase amplification, continued thermal cycling may force additional annealing to bead bound primers. Solution phase amplification may be minimal at this stage but concentration of immobilized B strands may increase. Then, the emulsion may be broken and the immobilized product may rendered single stranded by denaturing (by heat, pH, etc.) which may remove the complimentary A strand. The A primers may be annealed to the A′ region of an immobilized strand, and the immobilized strand may be loaded with sequencing enzymes, and any necessary accessory proteins. The beads may then be sequenced using recognized pyrophosphate techniques, such as those described in U.S. Pat. Nos. 6,274,320, 6258,568 and 6,210,891, the contents of which are incorporated herein by reference.

(1) Binding the Adaptor Primer to a Capture Bead

The adaptor primer may be attached to a capture bead. The primer may be attached to the solid support capture bead in any manner known in the art. Numerous methods exist in the art for attaching DNA to a solid support such as the microscopic bead. Covalent chemical attachment of the DNA to the bead may be accomplished by using standard coupling agents, such as water-soluble carbodiimide, to link the 5′-phosphate on the DNA to amine-coated capture beads through a phosphoamidate bond. Another alternative is to first couple specific oligonucleotide linkers to the bead using similar chemistry, and to then use DNA ligase to link the DNA to the linker on the bead. Other linkage chemistries to join the oligonucleotide to the beads include the use of N-hydroxysuccinamide (NHS) and its derivatives. In such a method, one end of the oligonucleotide may contain a reactive group (such as an amide group) which forms a covalent bond with the solid support, while the other end of the linker contains a second reactive group that can bond with the oligonucleotide to be immobilized. The oligonucleotide may be bound to the DNA capture bead by covalent linkage. However, non-covalent linkages, such as chelation or antigen-antibody complexes, may also be used to join the oligonucleotide to the bead.

Oligonucleotide linkers may be employed which specifically hybridize to unique sequences at the end of the DNA fragment, such as the overlapping end from a restriction enzyme site or the “sticky ends” of bacteriophage lambda based cloning vectors, but blunt-end ligations can also be used beneficially. These methods are described in detail in U.S. Pat. No. 5,674,743. The method used to immobilize the beads may continue to bind the immobilized oligonucleotide throughout the steps in the methods of the invention.

Each capture bead may be designed to have a plurality of primers that recognize (i.e., are complementary to) a portion of the isolated DNA, and the isolated DNA is thus hybridized to the capture bead. Only one unique isolated DNA may attached to any one capture bead in order to accomplish clonal amplification of the isolated DNA.

The beads used herein may be of any convenient size and fabricated from any number of known materials. Example of such materials include: inorganics, natural polymers, and synthetic polymers. Specific examples of these materials include: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and solid phase supports known to those of skill in the art. The capture beads may be Sepharose beads approximately 25 to 40 μm in diameter.

(2) Emulsification

Capture beads with attached single strand template nucleic acid may be emulsified as a heat stable water-in-oil emulsion. The emulsion may be formed according to any suitable method known in the art. One method of creating emulsion is described below but any method for making an emulsion may be used. These methods are known in the art and include adjuvant methods, counterflow methods, crosscurrent methods, rotating drum methods, and membrane methods. Furthermore, the size of the microcapsules may be adjusted by varying the flow rate and speed of the components. For example, in dropwise addition, the size of the drops and the total time of delivery may be varied. The emulsion may contain a density of bead “microreactors” at a density of about 3,000 beads per microliter.

The emulsion may be generated by suspending the template-attached beads in amplification solution. As used herein, the term “amplification solution” may mean the sufficient mixture of reagents that is necessary to perform amplification of template DNA.

The bead/amplification solution mixture may be added dropwise into a spinning mixture of biocompatible oil (e.g., light mineral oil, Sigma) and allowed to emulsify. The oil used may be supplemented with one or more biocompatible emulsion stabilizers. These emulsion stabilizers may include Atlox 4912, Span 80, and other recognized and commercially available suitable stabilizers. The droplets formed may range in size from 5 micron to 500 microns, from between about 50 to 300 microns, or from 100 to 150 microns.

There is no limitation in the size of the microreactors. The microreactors may be sufficiently large to encompass sufficient amplification reagents for the degree of amplification required. The microreactors may also be sufficiently small so that a population of microreactors, each containing a member of a DNA library, can be amplified by conventional laboratory equipment (e.g., PCR thermocycling equipment, test tubes, incubators and the like).

The optimal size of a microreactor may be between 100 to 200 microns in diameter. Microreactors of this size may allow amplification of a DNA library comprising about 600,000 members in a suspension of microreactors of less than 10 ml in volume. For example, if PCR was the chosen amplification method, 10 mls would fit in 96 tubes of a regular thermocycler with 96 tube capacity. The suspension of 600,000 microreactors may have a volume of less than 1 ml. A suspension of less than 1 ml may be amplified in about 10 tubes of a conventional PCR thermocycler. The suspension of 600,000 microreactors may also have a volume of less than 0.5 ml.

(3) Amplification

After encapsulation, the template nucleic acid may be amplified by any suitable method of DNA amplification including transcription-based amplification systems (Kwoh D. et al., Proc. Natl. Acad. Sci. (U.S.A.) 86:1173 (1989); Gingeras T. R. et al., PCT appl. WO 88/10315; Davey, C. et al., European Patent Application Publication No. 329,822; Miller, H. I. et al., PCT appl. WO 89/06700, and “race” (Frohman, M. A., In: PCR Protocols: A Guide to Methods and Applications, Academic Press, NY (1990)) and “one-sided PCR” (Ohara, O. et al., Proc. Natl. Acad. Sci. (U.S.A.) 86.5673-5677 (1989)). Still other less common methods such as “di-oligonucleotide” amplification, isothermal amplification (Walker, G. T. et al., Proc. Natl. Acad. Sci. (U.S.A.) 89:392-396 (1992)), and rolling circle amplification (reviewed in U.S. Pat. No. 5,714,320), may be used in the present invention.

DNA amplification may be performed by PCR. PCR may be performed by encapsulating the isolated DNA, bound to a bead, with a PCR solution comprising all the necessary reagents for PCR. Then, PCR may be accomplished by exposing the emulsion to any suitable thermocycling regimen known in the art. Between 30 and 50 cycles or about 40 cycles of amplification may be performed. Following the amplification procedure there may be one or more hybridization and extension cycles following the cycles of amplification. Between 10 and 30 cycles, or about 25 cycles of hybridization and extension may be performed. The template DNA may be amplified until typically at least two million to fifty million copies, or about ten million to thirty million copies of the template DNA are immobilized per bead.

(4) Breaking the Emulsion and Bead Recovery

Following amplification of the isolated DNA, the emulsion may be “broken” (also referred to as “demulsification”). There are many methods of breaking an emulsion, such as in U.S. Pat. No. 5,989,892, the contents of which are incorporated herein by reference. The emulsion may be broken by adding additional oil to cause the emulsion to separate into two phases. The oil phase may then be removed, and a suitable organic solvent (e.g., hexanes) may be added. After mixing, the oil/organic solvent phase is removed. This step may be repeated several times. Finally, the aqueous layers above the beads may be removed. The beads may then be washed with an organic solvent/annealing buffer mixture, and may then be washed again in annealing buffer. Suitable organic solvents include alcohols such as methanol, ethanol and the like.

The amplified isolated DNA-containing beads may then be resuspended in aqueous solution for use, for example, in a sequencing reaction according to known technologies. (See, Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, I. et al., Dokl Akad Nauk SSSR 303, 1508-1511 (1988); Bains W. & Smith G. C. J. TheorBiol 135, 303-307 (1988); Drnanac, R. et al., Genomics 4, 114-128 (1989); Khrapko, K. R. et al., FEBS Lett 256. 118-122 (1989); Pevzner P. A. J Biomol Struct Dyn 7, 63-73 (1989); Southern, E. M. et al., Genomics 13, 1008-1017 (1992).) The beads may be used in a pyrophosphate-based sequencing reaction (described, e.g., in U.S. Pat. Nos. 6,274,320, 6258,568 and 6,210,891, the contents of which are incorporated herein by reference), and the second strand of the PCR product may be removed, and a sequencing primer may be annealed to the single stranded isolated DNA that is bound to the bead.

The second strand may be melted away using any number of commonly known methods such as NaOH, low ionic (e.g., salt) strength, or heat processing. Following this melting step, the beads may be pelleted and the supernatant may be discarded. The beads may be resuspended in an annealing buffer, the sequencing primer may be added and may be annealed to the bead-attached single stranded template using a standard annealing cycle.

(5) Purifying the Beads

The amplified DNA on the bead may be sequenced either directly on the bead or in a different reaction vessel. The DNA may be sequenced directly on the bead by transferring the bead to a reaction vessel and subjecting the DNA to a sequencing reaction (e.g., pyrophosphate or Sanger sequencing). Alternatively, the beads may be isolated and the DNA may be removed from each bead and sequenced. In either case, the sequencing steps may be performed on each individual bead. The beads may also be purified according to a method described in U.S. Pat. No. 7,323,305, the contents of which are incorporated herein by reference.

b. Bridging PCR

The isolated DNA, which may be circular and may comprise the adaptor insert may be PCR amplified using “bridging” PCR. The bridging PCR may be performed according to methods described in U.S. Pat. No. 7,115,400, the contents of which are incorporated herein by reference. Prior to amplification, the isolated DNA may be linearized by digesting it with a restriction enzyme that cuts at a site between the two primer binding sites. The sequence at one end (the 5′ end) of the isolated DNA may have oligonucleotide sequence Y, and may comprise a sequence identical to the sequence of a “colony” primer X. Oligonucleotide sequence Y may be of a known sequence and may be of variable length. Oligonucleotide sequence Y may be at least five nucleotides in length, between 5 and 100 nucleotides in length, or approximately 20 nucleotides in length. Naturally occurring or non-naturally occurring nucleotides may be present in the oligonucleotide sequence Y.

The oligonucleotide sequence contained at the end of the isolated DNA opposite sequence Y (the 3′ end) may have oligonucleotide sequence Z. Oligonucleotide sequence Z may be of a known sequence and may be of variable length. Oligonucleotide sequence Z may be at least five nucleotides in length, between 5 and 100 nucleotides in length, or approximately 20 nucleotides in length. Naturally occurring or non-naturally occurring nucleotides may be present in the oligonucleotide sequence Z. Oligonucleotide sequence Z may be designed so that it hybridizes with colony primer λ and may be designed so that it is complementary to oligonucleotide sequence Y, referred to herein as Y′. The oligonucleotide sequences Y and Z contained at the 5′ and 3′ ends respectively of a nucleic acid template need not be located at the extreme ends of the template. For example although the oligonucleotide sequences Y and Z may be located at or near the 5′ and 3′ ends (or termini) respectively of the nucleic acid templates (for example within 0 to 100 nucleotides of the 5′ and 3′ termini) they may be located further away (e.g. greater than 100 nucleotides) from the 5′ or 3′ termini of the nucleic acid template. The oligonucleotide sequences Y and Z may therefore be located at any position within the nucleic acid template providing the sequences Y and Z are on either side, i.e. flank, the nucleic acid sequence which is to be amplified.

“Nucleic acid template” as used herein also includes an entity which comprises the nucleic acid to be amplified or sequenced in a double-stranded form. When the nucleic acid template is in a double-stranded form, the oligonucleotide sequences Y and Z are contained at the 5′ and 3′ ends respectively of one of the strands. The other strand, due to the base pairing rules of DNA, is complementary to the strand containing oligonucleotide sequences Y and Z and thus contains an oligonucleotide sequence Z′ at the 5′ end and an oligonucleotide sequence Y′ at the 3′ end.

“Colony primer” as used herein may refer to an entity which comprises an oligonucleotide sequence which is capable of hybridizing to a complementary sequence and initiate a specific polymerase reaction. The sequence comprising the colony primer is chosen such that it has maximal hybridizing activity with its complementary sequence and very low non-specific hybridizing activity to any other sequence. The sequence to be used as a colony primer can include any sequence, but may include 5′-AGAAGGAGAAGGAAAGGGAAAGGG (SEQ ID NO: 1) or 5′-CACCAACCCAAACCAACCCAAACC (SEQ ID NO: 2). The colony primer can be 5 to 100 bases in length, or 15 to 25 bases in length. Naturally occurring or non-naturally occurring nucleotides may be present in the primer. One or two different colony primers may be used to generate nucleic acid colonies in the methods of the present invention.

“Degenerate primer sequences” as used herein may refer to a short oligonucleotide sequence which is capable of hybridizing to any nucleic acid fragment independent of the sequence of said nucleic acid fragment. Such degenerate primers thus do not require the presence of oligonucleotide sequences Y or Z in the nucleic acid template(s) for hybridization to the template to occur, although the use of degenerate primers to hybridize to a template containing the oligonucleotide sequences λ or Y is not excluded. Clearly however, for use in the amplification methods of the present invention, the degenerate primers must hybridize to nucleic acid sequences in the template at sites either side, or flanking, the nucleic acid sequence which is to be amplified.

“Solid support” as used herein may refer to any solid surface to which nucleic acids can be covalently attached, such as for example latex beads, dextran beads, polystyrene, polypropylene surface, polyacrylamide gel, gold surfaces, glass surfaces and silicon wafers.

“Means for attaching nucleic acids to a solid support” as used herein may refer to any chemical or non-chemical attachment method including chemically-modifiable functional groups. “Attachment” relates to immobilization of nucleic acid on solid supports by either a covalent attachment or via irreversible passive adsorption or via affinity between molecules (for example, immobilization on an avidin-coated surface by biotinylated molecules). The attachment must be of sufficient strength that it cannot be removed by washing with water or aqueous buffer under DNA-denaturing conditions.

“Chemically-modifiable functional group” as used herein may refer to a group such as for example, a phosphate group, a carboxylic or aldehyde moiety, a thiol, or an amino group.

“Nucleic acid colony” as used herein may refer to a discrete area comprising multiple copies of a nucleic acid strand. Multiple copies of the complementary strand to the nucleic acid strand may also be present in the same colony. The multiple copies of the nucleic acid strands making up the colonies are generally immobilised on a solid support and may be in a single or double stranded form. The nucleic acid colonies of the invention can be generated in different sizes and densities depending on the conditions used. The size of colonies may be from 0.2 μm to 6 μm, or from 0.3 μm to 4 μm The density of nucleic acid colonies for use in the method of the invention may be from 10,000/mm² to 100,000/mm². It is believed that higher densities, for example, 100,000/mm² to 1,000,000/mm² and 1,000,000/mm² to 10,000,000/mm² may be achieved.

A nucleic acid colony may be generated from the isolated DNA. A plurality of colonies, each representing one of a plurality of different, isolated DNA may also be generated.

A plurality of isolated DNAs comprising the nucleic acids to be amplified may be generated, wherein the nucleic acids contain at their 5′ ends an oligonucleotide sequence Y and at the 3′ end an oligonucleotide sequence Z and, in addition, the nucleic acid(s) carry at the 5′ end a means for attaching the nucleic acid(s) to a solid support. The plurality of isolated DNAs is mixed with a plurality of colony primers λ which may hybridize to the oligonucleotide sequence Z and carry at the 5′ end a means for attaching the colony primers to a solid support. The plurality of isolated DNAs and colony primers may be covalently bound to a solid support.

Pluralities of two different colony primers λ may be mixed with the plurality of nucleic acid templates. The sequences of colony primers λ may be such that the oligonucleotide sequence Z can hybridize to one of the colony primers λ and the oligonucleotide sequence Y is the same as the sequence of one of the colony primers X.

The oligonucleotide sequence Z may also be complementary to oligonucleotide sequence Y, (Y′) and the plurality of colony primers λ may be of the same sequence as oligonucleotide sequence Y.

The plurality of colony primers λ may comprise a degenerate primer sequence and the plurality of isolated DNAs may comprise the nucleic acids to be amplified and may not contain oligonucleotide sequences Y or Z at the 5′ and 3′ ends respectively.

The oligonucleotide sequence contained at the 5′ end of the isolated nucleic acid may be of any sequence and any length and is denoted herein as sequence Y. Suitable lengths and sequences of oligonucleotide can be selected using methods well known and documented in the art. For example the oligonucleotide sequences attached to each end of the nucleic acid to be amplified are normally relatively short nucleotide sequences of between 5 and 100 nucleotides in length. The oligonucleotide sequence contained at the 3′ end of the nucleic acid can be of any sequence and any length and is denoted herein as sequence Z. Suitable lengths and sequences of oligonucleotide can be selected using methods well known and documented in the art. For example the oligonucleotide sequences contained at each end of the nucleic acid to be amplified are normally relatively short nucleotide sequences of between 5 and 100 nucleotides in length.

The sequence of the oligonucleotide sequence Z is such that it can hybridize to one of the colony primers X. The sequence of the oligonucleotide sequence Y may be such that it is the same as one of the colony primers X. The oligonucleotide sequence Z may be complementary to oligonucleotide sequence Y (Y′) and the colony primers λ are of the same sequence as oligonucleotide sequence Y.

The oligonucleotide sequences Y and Z may be prepared using techniques which are standard or conventional in the art, or may be purchased from commercial sources.

When producing the isolated DNAs of the invention additional desirable sequences can be introduced by methods well known and documented in the art. Such additional sequences include, for example, restriction enzyme sites or certain nucleic acid tags to enable amplification products of a given nucleic acid template sequence to be identified. Other desirable sequences include fold-back DNA sequences (which form hairpin loops or other secondary structures when rendered single-stranded), ‘control’ DNA sequences which direct protein/DNA interactions, such as for example a promoter DNA sequence which is recognised by a nucleic acid polymerase or an operator DNA sequence which is recognised by a DNA-binding protein.

If there are a plurality of nucleic acid sequences to be amplified then the attachment of oligonucleotides Y and Z can be carried out in the same or different reaction.

Once the isolated DNA has been prepared, it may be amplified before being used in the method described herein. Such amplification may be carried out using methods well known and documented in the art, for example by inserting the template nucleic acid into an expression vector and amplifying it in a suitable biological host, or amplifying it by PCR. This amplification step is not however essential, as the method of the invention allows multiple copies of the nucleic acid template to be produced in a nucleic acid colony generated from a single copy of the nucleic acid template.

The 5′ end of the isolated DNA may be modified to carry a means for attaching the nucleic acid templates covalently to a solid support. Such a means can be, for example, a chemically modifiable functional group, such as, for example a phosphate group, a carboxylic or aldehyde moiety, a thiol, or an amino group. The thiol, phosphate or amino group may be used for 5′-modification of the nucleic acid.

The colony primers may be prepared using techniques which are standard or conventional in the art. Generally, the colony primers of the invention will be synthetic oligonucleotides generated by methods well known and documented in the art or may be purchased from commercial sources.

One or two different colony primers X, may be used to amplify any nucleic acid sequence. The 5′ ends of colony primers λ may be modified to carry a means for attaching the colony primers covalently to the solid support. The covalent attachment may be a chemically modifiable functional group as described above. The colony primers may be designed to include additional desired sequences such as, for example, restriction endonuclease sites or other types of cleavage sites each as ribozyme cleavage sites. The additional sequences include fold-back DNA sequences (which form hairpin loops or other secondary structures when rendered single-stranded), ‘control’ DNA sequences which direct a protein/DNA interaction, such as for example a promoter DNA sequence which is recognised by a nucleic acid polymerase or an operator DNA sequence which is recognised by a DNA-binding protein.

Immobilisation of a colony primer λ to a support by the 5′ end may leave its 3′ end remote from the support such that the colony primer is available for chain extension by a polymerase once hybridization with a complementary oligonucleotide sequence contained at the 3′ end of the isolated DNA has taken place.

Once both the isolated DNA and colony primers of the invention have been synthesised they are mixed together in appropriate proportions so that when they are attached to the solid support an appropriate density of attached isolated DNA and colony primers is obtained. The proportion of colony primers in the mixture may be higher than the proportion of isolated DNA. The ratio of colony primers to isolated DNA may be such that when the colony primers and isolated DNA are immobilised to the solid support a “lawn” of colony primers is formed comprising a plurality of colony primers being located at an approximately uniform density over the whole or a defined area of the solid support, with one or more isolated DNAs being immobilised individually at intervals within the lawn of colony primers.

The isolated DNA may be provided in single stranded form. However, it may also be provided totally or partly in double stranded form, either with one 5′ end or both 5′ ends modified so as to allow attachment to the support. In that case, after completion of the attachment process, one might want to separate strands by means known in the art, e.g. by heating to 94° C., before washing the released strands away. Where both strands of the double stranded molecules have reacted with the surface and are both attached, the result may be the same as in the case when only one strand is attached and one amplification step has been performed. In other words, in the case where both strands of a double stranded isolated DNA have been attached, both strands are necessarily attached close to each other and are indistinguishable from the result of attaching only one strand and performing one amplification step. Thus, single stranded and double stranded isolated DNA might be used for providing template nucleic acids attached to the surface and suitable for colony generation.

The distance between the individual colony primers and the individual isolated DNA (and hence the density of the colony primers and isolated DNA) can be controlled by altering the concentration of colony primers and isolated DNA that are immobilised to the support. The density of colony primers may be at least 1 fmol/mm², at least 10 fmol/mm², or between 30 to 60 fmol/mm². The density of isolated DNA may be 10,000/mm² to 100,000/mm². Higher densities of 100,000/mm² to 1,000,000/mm² and 1,000,000/mm² to 10,000,000/mm² may be achieved.

Controlling the density of attached isolated DNA and colony primers may allow the final density of nucleic acid colonies on the surface of the support to be controlled. This is due to the fact that according to the method of the invention, one nucleic acid colony can result from the attachment of one isolated DNA, providing the colony primers are present in a suitable location on the solid support. The density of isolated DNA within a single colony can also be controlled by controlling the density of attached colony primers.

Once the colony primers and isolated DNA have been immobilised on the solid support at the appropriate density, nucleic acid colonies mas be generated by carrying out an appropriate number of cycles of amplification on the covalently bound isolated DNA so that each colony comprises multiple copies of the original immobilised isolated DNA and its complementary sequence. One cycle of amplification consists of the steps of hybridization, extension and denaturation and these steps are generally performed using reagents and conditions well known in the art for PCR.

A typical amplification reaction comprises subjecting the solid support and attached isolated DNA and colony primers to conditions which induce primer hybridization, for example subjecting them to a temperature of around 65° C. Under these conditions the oligonucleotide sequence Z at the 3′ end of the isolated DNA will hybridize to the immobilised colony primer X and in the presence of conditions and reagents to support primer extension, for example a temperature of around 72° C., the presence of a nucleic acid polymerase, for example, a DNA dependent DNA polymerase or a reverse transcriptase molecule (i.e. an RNA dependent DNA polymerase), or an RNA polymerase, plus a supply of nucleoside triphosphate molecules or any other nucleotide precursors, for example modified nucleoside triphosphate molecules, the colony primer will be extended by the addition of nucleotides complementary to the isolated DNA.

The nucleic acid polymerase may be DNA polymerase (Klenow fragment, T4 DNA polymerase), a heat-stable DNA polymerase from a thermostable bacteria (such as Taq, VENT, Pfu, Tfl DNA polymerases), or a genetically modified derivative thereof (TaqGold, VENTexo, Pfu exo). A combination of RNA polymerase and reverse transcriptase may also be used to generate the amplification of a DNA colony. The nucleic acid polymerase used for colony primer extension may be stable under PCR reaction conditions, i.e., repeated cycles of heating and cooling, and may be stable at the denaturation temperature used, usually approximately 94° C.

The nucleoside triphosphate molecules may be deoxyribonucleotide triphosphates, for example DATP, dTTP, dCTP, dGTP, or are ribonucleoside triphosphates for example dATP, dUTP, dCTP, dGTP. The nucleoside triphosphate molecules may be naturally or non-naturally occurring.

After the hybridization and extension steps, on subjecting the support and attached nucleic acids to denaturation conditions, two immobilised nucleic acids will be present, the first being the initial immobilised isolated DNA and the second being a nucleic acid complementary thereto, extending from one of the immobilised colony primers X. Both the original immobilised isolated DNA and the immobilised extended colony primer formed are then able to initiate further rounds of amplification on subjecting the support to further cycles of hybridization, extension and denaturation. Such further rounds of amplification will result in a nucleic acid colony comprising multiple immobilised copies of the isolated DNA and its complementary sequence.

The initial immobilisation of the isolated DNA means that the template nucleic acid can only hybridize with colony primers located at a distance within the total length of the isolated DNA. Thus the boundary of the nucleic acid colony formed is limited to a relatively local area to the area in which the initial isolated DNA was immobilised. Clearly, once more copies of the isolated DNA and its complement have been synthesised by carrying out further rounds of amplification, i.e.l, further rounds of hybridization, extension and denaturation, then the boundary of the nucleic acid colony being generated will be able to be extended further, although the boundary of the colony formed may still limited to a relatively local area to the area in which the initial isolated DNA was immobilised.

A nucleic acid colony from a single immobilised isolated DNA may be generated, and the size of these colonies can be controlled by altering the number of rounds of amplification that the isolated DNA is subjected to. Thus the number of nucleic acid colonies formed on the surface of the solid support is dependent upon the number of isolated DNA which is initially immobilised to the support, providing there is a sufficient number of immobilised colony primers within the locality of each immobilised isolated DNA. It is for this reason that the solid support to which the colony primers and isolated DNA have been immobilised may comprise a lawn of immobilised colony primers at an appropriate density with isolated DNA immobilised at intervals within the lawn of primers.

Such so called “autopatterning” of nucleic acid colonies may have an advantage over other methods in that a higher density of nucleic acid colonies can be obtained due to the fact that the density can be controlled by regulating the density at which the isolated DNA are originally immobilised. Such a method is thus not limited by, for example, having specifically to array specific primers on particular local areas of the support and then initiate colony formation by spotting a particular sample containing isolated DNA on the same local area of primer. The numbers of colonies that can be arrayed using prior art methods, for example those disclosed in WO96/04404 (Mosaic Technologies, Inc.) is thus limited by the density/spacing at which the specific primer areas can be arrayed in the initial step.

By being able to control the initial density of the isolated DNA and hence the density of the nucleic acid colonies resulting from the isolated DNA, together with being able to control the size of the nucleic acid colonies formed and in addition the density of the isolated DNA within individual colonies, an optimum situation can be reached wherein a high density of individual nucleic acid colonies can be produced on a solid support of a large enough size and containing a large enough number of amplified sequences to enable subsequent analysis or monitoring to be performed on the nucleic acid colonies.

Once nucleic acid colonies have been generated it may be desirable to carry out an additional step such as for example colony visualisation or sequence determination. Colony visualisation might for example be required if it was necessary to screen the colonies generated for the presence or absence of for example the whole or part of a particular nucleic acid fragment. In this case the colony or colonies which contain the particular nucleic acid fragment could be detected by designing a nucleic acid probe which specifically hybridizes to the nucleic acid fragment of interest.

Such a nucleic acid probe may be labeled with a detectable entity such as a fluorescent group, a biotin containing entity (which can be detected by for example an incubation with streptavidin labeled with a fluorescent group), a radiolabel (which can be incorporated into a nucleic acid probe by methods well known and documented in the art and detected by detecting radioactivity for example by incubation with scintillation fluid), or a dye or other staining agent.

The nucleic acid probe may also be unlabeled and designed to act as a primer for the incorporation of a number of labeled nucleotides with a nucleic acid polymerase. Detection of the incorporated label and thus the nucleic acid colonies can then be carried out.

The nucleic acid colonies may then be prepared for hybridization. Such preparation may involve the treatment of the colonies so that all or part of the nucleic acid templates making up the colonies is present in a single stranded form. This can be achieved for example by heat denaturation of any double stranded DNA in the colonies. Alternatively the colonies may be treated with a restriction endonuclease specific for a double stranded form of a sequence in the template nucleic acid. Thus the endonuclease may be specific for either a sequence contained in the oligonucleotide sequences Y or Z or another sequence present in the isolated DNA. After digestion the colonies are heated so that double stranded DNA molecules are separated and the colonies are washed to remove the non-immobilised strands thus leaving attached single stranded DNA in the colonies.

After preparation of the colonies for hybridization, the labeled or unlabeled probe may then added to the colonies under conditions appropriate for the hybridization of the probe with its specific DNA sequence.

The probe may then be removed by heat denaturation and, if desired, a probe specific for a second nucleic acid may be hybridized and detected. These steps may be repeated as many times as is desired.

Labeled probes which are hybridized to nucleic acid colonies may then be detected using apparatus including an appropriate detection device. The detection system for fluorescent labels may be a charge-coupled device (CCD) camera, which can optionally be coupled to a magnifying device, for example a microscope. Using such technology it is possible to simultaneously monitor many colonies in parallel. For example, using a microscope with a CCD camera and a 10× or 20× objective it is possible to observe colonies over a surface of between 1 mm² and 4 mm², which corresponds to monitoring between 10 000 and 200 000 colonies in parallel.

An alternative method of monitoring the colonies generated is to scan the surface covered with colonies. For example systems in which up to 100 000 000 colonies could be arrayed simultaneously and monitored by taking pictures with the CCD camera over the whole surface can be used. In this way, it can be seen that up to 100 000 000 colonies could be monitored in a short time.

Any other devices allowing detection and quantification of fluorescence on a surface may be used to monitor the nucleic acid colonies of the invention. For example fluorescent imagers or confocal microscopes could be used. If the labels are radioactive then a radioactivity detection system may be required.

The sequence of the isolated DNA may be determined by using any appropriate solid phase sequencing technique. For example, one technique of sequence determination that may be used in the present invention involves hybridizing an appropriate primer, sometimes referred to herein as a “sequencing primer”, with the nucleic acid template to be sequenced, extending the primer and detecting the nucleotides used to extend the primer. The nucleic acid used to extend the primer may be detected before a further nucleotide is added to the growing nucleic acid chain, thus allowing base by base in situ nucleic acid sequencing.

The detection of incorporated nucleotides may be facilitated by including one or more labeled nucleotides in the primer extension reaction. Any appropriate detectable label may be used, for example a fluorophore, radiolabel etc. A fluorescent label may be used. The same or different labels may be used for each different type of nucleotide. Where the label is a fluorophore and the same labels are used for each different type of nucleotide, each nucleotide incorporation may provide a cumulative increase in signal detected at a particular wavelength. If different labels are used then these signals may be detected at different appropriate wavelengths. If desired a mixture of labeled and unlabeled nucleotides are provided.

In order to allow the hybridization of an appropriate sequencing primer to the isolated DNA to be sequenced the nucleic acid template may be in a single stranded form. If the nucleic acid templates making up the nucleic acid colonies are present in a double stranded form these can be processed to provide single stranded isolated DNA using methods well known in the art, for example by denturation, cleavage etc.

The sequencing primers which are hybridized to the isolated DNA and used for primer extension may be short oligonucleotides, for example of 15 to 25 nucleotides in length. The sequence of the primers may be designed so that they hybridize to part of the isolated DNA to be sequenced, and may be under stringent conditions. The sequence of the primers used for sequencing may have the same or similar sequences to that of the colony primers used to generate the nucleic acid colonies. The sequencing primers may be provided in solution or in an immobilised form.

Once the sequencing primer has been annealed to the isolated DNA to be sequenced by subjecting the isolated DNA and sequencing primer to appropriate conditions, determined by methods well known in the art, primer extension may be carried out, for example using a nucleic acid polymerase and a supply of nucleotides, at least some of which are provided in a labeled form, and conditions suitable for primer extension if a suitable nucleotide is provided.

After each primer extension step, a washing step may be included in order to remove unincorporated nucleotides which may interfere with subsequent steps. Once the primer extension step has been carried out the nucleic acid colony may be monitored in order to determine whether a labeled nucleotide has been incorporated into an extended primer. The primer extension step may then be repeated in order to determine the next and subsequent nucleotides incorporated into an extended primer.

Any device allowing detection and quantification of the appropriate label, for example fluorescence or radioactivity, may be used for sequence determination. If the label is fluorescent a CCD camera optionally attached to a magnifying device, may be used. In fact the devices used for sequencing may be the same as those described above for monitoring the amplified nucleic acid colonies.

The detection system may be used in combination with an analysis system in order to determine the number and nature of the nucleotides incorporated at each colony after each step of primer extension. This analysis, which may be carried out immediately after each primer extension step, or later using recorded data, allows the sequence of the nucleic acid template within a given colony to be determined.

If the sequence being determined is unknown, the nucleotides applied to a given colony may be applied in a chosen order which is then repeated throughout the analysis, for example dATP, dTTP, dCTP, dGTP. If however, the sequence being determined is known and is being resequenced, for example to analyse whether or not small differences in sequence from the known sequence are present, the sequencing determination process may be made quicker by adding the nucleotides at each step in the appropriate order, chosen according to the known sequence. Differences from the given sequence are thus detected by the lack of incorporation of certain nucleotides at particular stages of primer extension.

The attachment of the colony primer and nucleic acid template to the solid support may be thermostable at the temperature to which the support may be subjected to during the nucleic acid amplification reaction, for example temperatures of up to approximately 100° C., for example approximately 94° C. The attachment may be covalent, and may be accomplished as described in U.S. Pat. No. 7,115,400, the contents of which are incorporated herein by reference.

5. Sequencing the Isolated DNA

The sequencing may be performed by methods described in Intl. Pub. Nos. WO 2007/145612 or WO 05003375, or U.S. Pat. No. 7,323,305 or 7,115,400, the contents of which are incorporated herein by reference. The machine may be a Roche (454) GS FLX (454 Life Systems, Branford, Conn.), an Illumina Genome Analyzer sequencing primer (Illumina Inc., San Diego, Calif.).

For example, pyrophosphate sequencing may be used. This technique is based on the detection of released pyrophosphate (Ppi) during DNA synthesis, as described in Hyman, 1988. A new method of sequencing DNA. Anal Biochem. 174:423-36; and Ronaghi, 2001. Pyrosequencing sheds light on DNA sequencing. Genome Res. 11:3-11.

In a cascade of enzymatic reactions, visible light may be generated proportional to the number of incorporated nucleotides. The cascade may start with a nucleic acid polymerization reaction in which inorganic Ppi may be released with nucleotide incorporation by polymerase. The released Ppi may be converted to ATP by ATP sulfurylase, which may provide the energy to luciferase to oxidize luciferin and may generate light. Because the added nucleotide is known, the sequence of the template may be determined. Solid-phase pyrophosphate sequencing utilizes immobilized DNA in a three-enzyme system. To increase the signal-to-noise ratio, the natural dATP may be replaced by dATPαS. dATPαS may be a mixture of two isomers (Sp and Rp). Pure 2′-deoxyadenosine-5′-O′-(1-thiotriphosphate) Sp-isomer in pyrophosphate may be used in sequencing to allow substantially longer reads, up to doubling of the read length.

Pyrophosphate-based sequencing may be performed by subjecting the isolated DNA and the extension primer to a polymerase reaction in the presence of a nucleotide triphosphate whereby the nucleotide triphosphate will only become incorporated and release pyrophosphate (PPi) if it is complementary to the base in the target position, the nucleotide triphosphate being added either to separate aliquots of sample-primer mixture or successively to the same sample-primer mixture. The release of PPi maybe then be detected to indicate which nucleotide is incorporated.

A region of the sequence product may be determined by annealing a sequencing primer to a region of the isolated DNA, and then contacting the sequencing primer with a DNA polymerase and a known nucleotide triphosphate, i.e., dATP, dCTP, dGTP, dTTP, or an analog of one of these nucleotides. The sequence may be determined by detecting a sequence reaction byproduct, as is described below.

The sequence primer can be any length or base composition, as long as it is capable of specifically annealing to a region of the amplified nucleic acid template. No particular structure for the sequencing primer is required so long as it is able to specifically prime a region on the amplified template nucleic acid. The sequencing primer may complementary to a region of the template that is between the sequence to be characterized and the sequence hybridizable to the anchor primer. The sequencing primer is extended with the DNA polymerase to form a sequence product. The extension is performed in the presence of one or more types of nucleotide triphosphates, and if desired, auxiliary binding proteins.

Incorporation of the dNTP may be determined by assaying for the presence of a sequencing byproduct. The nucleotide sequence of the sequencing product may also be determined by measuring inorganic pyrophosphate (PPi) liberated from a nucleotide triphosphate (dNTP) as the dNMP is incorporated into an extended sequence primer. This method of sequencing, termed Pyrosequencing™ technology (PyroSequencing AB, Stockholm, Sweden) may be performed in solution (liquid phase) or as a solid phase technique. PPi-based sequencing methods are described generally in, e.g., WO9813523A1, Ronaghi, et al., 1996. Anal. Biochem. 242: 84-89, Ronaghi, et al., 1998. Science 281: 363-365 (1998) and USSN 2001/0024790, the contents of which are incorporated herein by reference. See also, e.g., U.S. Pat. Nos. 6,210,891 and 6,258,568, the contents of which incorporated herein by reference.

Pyrophosphate released under these conditions may be detected enzymatically (e.g., by the generation of light in the luciferase-luciferin reaction). Such methods may enable a nucleotide to be identified in a given target position, and the DNA to be sequenced simply and rapidly while avoiding the need for electrophoresis and the use of potentially dangerous radiolabels.

PPi may be detected by a number of different methodologies, and various enzymatic methods have been previously described (see e.g., Reeves, et al., 1969. Anal. Biochem. 28: 282-287; Guillory, et al., 1971. Anal. Biochem. 39: 170-180; Johnson, et al., 1968. Anal. Biochem. 15: 273; Cook, et al., 1978. Anal. Biochem. 91: 557-565; and Drake, et al., 1979. Anal. Biochem. 94: 117-120).

PPi liberated as a result of incorporation of a dNTP by a polymerase may be converted to ATP using, e.g., an ATP sulfurylase. This enzyme has been identified as being involved in sulfur metabolism. Sulfur, in both reduced and oxidized forms, is an essential mineral nutrient for plant and animal growth (see e.g., Schmidt and Jager, 1992. Ann. Rev. Plant Physiol. Plant Mol. Biol. 43: 325-349). In both plants and microorganisms, active uptake of sulfate is followed by reduction to sulfide. As sulfate has a very low oxidation/reduction potential relative to available cellular reductants, the primary step in assimilation requires its activation via an ATP-dependent reaction (see e.g., Leyh, 1993. Crit. Rev. Biochem. Mol. Biol. 28: 515-542). ATP sulfurylase (ATP: sulfate adenylyltransferase; EC 2.7.7.4) catalyzes the initial reaction in the metabolism of inorganic sulfate (SO.sub.4.sup.-2); see e.g., Robbins and Lipmann, 1958. J. Biol. Chem. 233: 686-690; Hawes and Nicholas, 1973. Biochem. J. 133: 541-550). In this reaction SO.sub.4.sup.-2 is activated to adenosine 5′-phosphosulfate (APS).

ATP sulfurylase has been highly purified from several sources, such as Saccharomyces cerevisiae (see e.g., Hawes and Nicholas, 1973. Biochem. J. 133: 541-550); Penicillium chrysogenum (see e.g., Renosto, et al., 1990. J. Biol. Chem. 265: 10300-10308); rat liver (see e.g., Yu, et al., 1989. Arch. Biochem. Biophys. 269: 165-174); and plants (see e.g., Shaw and Anderson, 1972. Biochem. J. 127: 237-247; Osslund, et al., 1982. Plant Physiol. 70: 39-45). Furthermore, ATP sulfurylase genes have been cloned from prokaryotes (see e.g., Leyh, et al., 1992. J. Biol. Chem. 267: 10405-10410; Schwedocki and Long, 1989. Mol. Plant. Microbe Interaction 2: 181-194; Laue and Nelson, 1994. J. Bacteriol. 176: 3723-3729); eukaryotes (see e.g., Cherest, et al., 1987. Mol. Gen. Genet. 210: 307-313; Mountain and Korch, 1991. Yeast 7: 873-880; Foster, et al., 1994. J. Biol. Chem. 269: 19777-19786); plants (see e.g., Leustek, et al., 1994. Plant Physiol. 105: 897-90216); and animals (see e.g., Li, et al., 1995. J. Biol. Chem. 270: 29453-29459). The enzyme is a homo-oligomer or heterodimer, depending upon the specific source (see e.g., Leyh and Suo, 1992. J. Biol. Chem. 267: 542-545).

A thermostable sulfurylase may be used. The thermostable sulfurylase may be obtained from, e.g., Archaeoglobus or Pyrococcus spp. Sequences of thermostable sulfurylases are available at database Acc. No. 028606, Acc. No. Q9YCR4, and Acc. No. P56863.

ATP sulfurylase has been used for many different applications, for example, bioluminometric detection of ADP at high concentrations of ATP (see e.g., Schultz, et al., 1993. Anal. Biochem. 215: 302-304); continuous monitoring of DNA polymerase activity (see e.g., Nyrbn, 1987. Anal. Biochem. 167: 235-238); and DNA sequencing (see e.g., Ronaghi, et al., 1996. Anal. Biochem. 242: 84-89; Ronaghi, et al., 1998. Science 281: 363-365; Ronaghi, et al., 1998. Anal. Biochem. 267: 65-71).

Several assays have been developed for detection of the forward ATP sulfurylase reaction. The colorimetric molybdolysis assay may be based on phosphate detection (see e.g., Wilson and Bandurski, 1958. J. Biol. Chem. 233: 975-981), whereas the continuous spectrophotometric molybdolysis assay may based upon the detection of NADH oxidation (see e.g., Seubert, et al., 1983. Arch. Biochem. Biophys. 225: 679-691; Seubert, et al., 1985. Arch. Biochem. Biophys. 240: 509-523). The later assay may require the presence of several detection enzymes. In addition, several radioactive assays have also been described in the literature (see e.g., Daley, et al., 1986. Anal. Biochem. 157: 385-395). For example, one assay is based upon the detection of ³²PPi released from ³²P-labeled ATP (see e.g., Seubert, et al., 1985. Arch. Biochem. Biophys. 240: 509-523) and another on the incorporation of ³⁵S into [³⁵5]-labeled APS (this assay also requires purified APS kinase as a coupling enzyme; see e.g., Seubert, et al., 1983. Arch. Biochem. Biophys. 225: 679-691); and a third reaction depends upon the release of ³⁵SO₄⁻² from ³⁵-labeled APS (see e.g., Daley, et al., 1986. Anal. Biochem. 157: 385-395).

For detection of the reversed ATP sulfurylase reaction a continuous spectrophotometric assay (see e.g., Segel, et al., 1987. Methods Enzymol. 143: 334-349); a bioluminometric assay (see e.g., Balharry and Nicholas, 1971. Anal. Biochem. 40:1-17); an ³⁵SO₄⁻² release assay (see e.g., Seubert, et al., 1985. Arch. Biochem. Biophys. 240: 509-523); or a ³²PPi incorporation assay (see e.g., Osslund, et al., 1982. Plant Physiol. 70: 39-45) may be used.

ATP produced by an ATP sulfurylase may be hydrolyzed using enzymatic reactions to generate light. Light-emitting chemical reactions (i.e., chemiluminescence) and biological reactions (i.e., bioluminescence) are widely used in analytical biochemistry for sensitive measurements of various metabolites. In bioluminescent reactions, the chemical reaction that leads to the emission of light may be enzyme-catalyzed. For example, the luciferin-luciferase system allows for specific assay of ATP and the bacterial luciferase-oxidoreductase system may be used for monitoring of NAD(P)H. Both systems have been extended to the analysis of numerous substances by means of coupled reactions involving the production or utilization of ATP or NAD(P)H (see e.g., Kricka, 1991. Chemiluminescent and bioluminescent techniques. Clin. Chem. 37: 1472-1281).

The development of new reagents have made it possible to obtain stable light emission proportional to the concentrations of ATP (see e.g., Lundin, 1982. Applications of firefly luciferase In; Luminescent Assays (Raven Press, New York) or NAD(P)H (see e.g., Lovgren, et al., Continuous monitoring of NADH-converting reactions by bacterial luminescence. J. Appl. Biochem. 4: 103-111). With such stable light emission reagents, it is possible to make endpoint assays and to calibrate each individual assay by addition of a known amount of ATP or NAD(P)H. In addition, a stable light-emitting system also allows continuous monitoring of ATP- or NAD(P)H-converting systems.

Suitable enzymes for converting ATP into light include luciferases, e.g., insect luciferases. Luciferases produce light as an end-product of catalysis. The best known light-emitting enzyme is that of the firefly, Photinus pyralis (Coleoptera). The corresponding gene has been cloned and expressed in bacteria (see e.g., de Wet, et al., 1985. Proc. Natl. Acad. Sci. USA 80: 7870-7873) and plants (see e.g., Ow, et al., 1986. Science 234: 856-859), as well as in insect (see e.g., Jha, et al., 1990. FEBS Lett. 274: 24-26) and mammalian cells (see e.g., de Wet, et al., 1987. Mol. Cell. Biol. 7: 725-7373; Keller, et al., 1987. Proc. Natl. Acad. Sci. USA 82: 3264-3268). In addition, a number of luciferase genes from the Jamaican click beetle, Pyroplorus plagiophihalamus (Coleoptera), have recently been cloned and partially characterized (see e.g., Wood, et al., 1989. J. Biolumin. Chemilumin. 4: 289-301; Wood, et al., 1989. Science 244: 700-702). Distinct luciferases can sometimes produce light of different wavelengths, which may enable simultaneous monitoring of light emissions at different wavelengths. Accordingly, these aforementioned characteristics are unique, and add new dimensions with respect to the utilization of current reporter systems.

Firefly luciferase may catalyze bioluminescence in the presence of luciferin, adenosine 5′-triphosphate (ATP), magnesium ions, and oxygen, resulting in a quantum yield of 0.88 (see e.g., McElroy and Selinger, 1960. Arch. Biochem. Biophys. 88: 136-145). The firefly luciferase bioluminescent reaction can be utilized as an assay for the detection of ATP with a detection limit of approximately 1.times.10.sup.-13 M (see e.g., Leach, 1981. J. Appl. Biochem. 3: 473-517). In addition, the overall degree of sensitivity and convenience of the luciferase-mediated detection systems have created considerable interest in the development of firefly luciferase-based biosensors (see e.g., Green and Kricka, 1984. Talanta 31: 173-176; Blum, et al., 1989. J. Biolumin. Chemilumin. 4: 543-550).

Using the above-described enzymes, the sequence primer may be exposed to a polymerase and a known dNTP. If the dNTP is incorporated onto the 3′ end of the primer sequence, the dNTP may be cleaved and a PPi molecule may be liberated. The PPi may then be converted to ATP with ATP sulfurylase. The ATP sulfurylase may be present at a sufficiently high concentration that the conversion of PPi proceeds with first-order kinetics with respect to PPi. In the presence of luciferase, the ATP is hydrolyzed to generate a photon. The reaction may have a sufficient concentration of luciferase present within the reaction mixture such that the reaction, ATPDP→DP+PO₄³⁻+photon (light), proceeds with first-order kinetics with respect to ATP. The photon may be measured using methods and apparatuses described below. The PPi and a coupled sulfurylase/luciferase reaction may be used to generate light for detection. Either or both the sulfurylase and luciferase may be immobilized on one or more mobile solid supports disposed at each reaction site.

PPi may be released to be detected during the polymerase reaction giving a real-time signal. The sequencing reactions may be continuously monitored in real-time. The reactions may take place in less than 2 seconds (Nyren and Lundin, supra). The rate limiting step may be the conversion of PPi to ATP by ATP sulftirylase, while the luciferase reaction is fast and has been estimated to take less than 0.2 seconds. Incorporation rates for polymerases have also been estimated by various methods and it has been found, for example, that in the case of Klenow polymerase, complete incorporation of one base may take less than 0.5 seconds. Thus, the estimated total time for incorporation of one base and detection by this enzymatic assay is approximately 3 seconds. It will be seen therefore that very fast reaction times are possible, enabling real-time detection. The reaction times could further be decreased by using a more thermostable luciferase.

For most applications reagents free of contaminants like ATP and PPi may be used. These contaminants may be removed by flowing the reagents through a pre-column containing apyrase and/-or pyrophosphatase bound to resin. Alternatively, the apyrase or pyrophosphatase may be bound to magnetic beads and used to remove contaminating ATP and PPi present in the reagents. In addition diffusible sequencing reagents, e.g., unincorporated dNTPs, may be washed away with a wash buffer. Any wash buffer used in pyrophosphate sequencing may be used.

The concentration of reactants in the sequencing reaction may include 1 pmol DNA, 3 pmol polymerase, 40 pmol dNTP in 0.2 ml buffer. See Ronaghi, et al., Anal. Biochem. 242: 84-89 (1996).

The sequencing reaction may be performed with each of four predetermined nucleotides, if desired. A “complete” cycle may include sequentially administering sequencing reagents for each of the nucleotides DATP, dGTP, dCTP and dTTP (or dUTP), in a predetermined order. Unincorporated dNTPs may be washed away between each of the nucleotide additions. Alternatively, unincorporated dNTPs may be degraded by apyrase. The cycle may be repeated as desired until the desired amount of sequence of the sequence product is obtained. About 10-1000, 10-100, 10-75, 20-50, or about 30 nucleotides of sequence information may be obtained from extension of one annealed sequencing primer.

The nucleotide may be modified to contain a disulfide-derivative of a hapten such as biotin. The addition of the modified nucleotide to the nascent primer annealed to the anchored substrate may be analyzed by a post-polymerization step that includes i) sequentially binding of, in the example where the modification is a biotin, an avidin- or streptavidin-conjugated moiety linked to an enzyme molecule, ii) the washing away of excess avidin- or streptavidin-linked enzyme, iii) the flow of a suitable enzyme substrate under conditions amenable to enzyme activity, and iv) the detection of enzyme substrate reaction product or products. The hapten may be removed through the addition of a reducing agent. Such methods may enable a nucleotide to be identified in a given target position, and the DNA to be sequenced simply and rapidly while avoiding the need for electrophoresis and the use of potentially dangerous radiolabels.

The enzyme for detecting the hapten may be horse-radish peroxidase. A wash buffer may be used between the addition of various reactants herein. Apyrase may be used to remove unreacted dNTP used to extend the sequencing primer. The wash buffer may include apyrase.

Example haptens, e.g., biotin, digoxygenin, the fluorescent dye molecules cy3 and cy5, and fluorescein, may be incorporated at various efficiencies into extended DNA molecules. The attachment of the hapten may occur through linkages via the sugar, the base, and via the phosphate moiety on the nucleotide. Example means for signal amplification include fluorescent, electrochemical and enzymatic. If enzymatic amplification is used, the enzyme, e.g. alkaline phosphatase (AP), horse-radish peroxidase (HRP), beta-galactosidase, luciferase, may include those for which light-generating substrates are known, and the means for detection of these light-generating (chemiluminescent) substrates may include a CCD camera.

The modified base may added, detection may occur, and the hapten-conjugated moiety may be removed or inactivated by use of either a cleaving or inactivating agent. For example, if the cleavable-linker is a disulfide, then the cleaving agent may be a reducing agent, for example dithiothreitol (DTT), beta-mercaptoethanol, etc. Inactivation may also be accomplished by heat, cold, chemical denaturant, surfactant, hydrophobic reagent, or a suicide inhibitor.

Luciferase may hydrolyze dATP directly with concomitant release of a photon. This may result in a false positive signal because the hydrolysis occurs independent of incorporation of the dATP into the extended sequencing primer. To avoid this problem, a dATP analog may be used which is incorporated into DNA, i.e., it is a substrate for a DNA polymerase, but is not a substrate for luciferase. One such analog is α-thio-dATP. Thus, use of α-thio-dATP may avoid the spurious photon generation that can occur when dATP is hydrolyzed without being incorporated into a growing nucleic acid chain.

The PPi-based detection may calibrated by the measurement of the light released following the addition of control nucleotides to the sequencing reaction mixture immediately after the addition of the sequencing primer. This may allow for normalization of the reaction conditions. Incorporation of two or more identical nucleotides in succession may be revealed by a corresponding increase in the amount of light released. Thus, a two-fold increase in released light relative to control nucleotides may reveal the incorporation of two successive dNTPs into the extended primer.

Apyrase may be “washed” or “flowed” over the surface of the solid support so as to facilitate the degradation of any remaining, non-incorporated dNTPs within the sequencing reaction mixture. Apyrase may also degrade the generated ATP and hence “turns off” the light generated from the reaction. Upon treatment with apyrase, any remaining reactants may be washed away in preparation for the following dNTP incubation and photon detection steps. Alternatively, the apyrase may be bound to the solid or mobile solid support.

a. Detecting the Sequencing Reaction

The solid support may be optically linked to an imaging system 230, which may include a CCD system in association with conventional optics or a fiber optic bundle. The perfusion chamber substrate may include a fiber optic array wafer such that light generated near the aqueous interface may be transmitted directly through the optical fibers to the exterior of the substrate or chamber. When the CCD system includes a fiber optic connector, imaging may be accomplished by placing the perfusion chamber substrate in direct contact with the connector. Alternatively, conventional optics may be used to image the light, e.g., by using a 1-1 magnification high numerical aperture lens system, from the exterior of the fiber optic substrate directly onto the CCD sensor. When the substrate does not provide for fiber optic coupling, a lens system may also be used as described above, in which case either the substrate or the perfusion chamber cover is optically transparent.

The imaging system 230 may be used to collect light from the reactors on the substrate surface. Light may be imaged, for example, onto a CCD using a high sensitivity low noise apparatus known in the art. For fiber-optic based imaging, the optical fibers may be incorporated directly into the cover slip or for a FORA to have the optical fibers that form the microwells also be the optical fibers that convey light to the detector.

The imaging system may be linked to a computer control and data collection system 240. Any commonly available hardware and software package may be used. The computer control and data collection system may also be linked to the conduit 200 to control reagent delivery.

The photons generated by the pyrophosphate sequencing reaction may be captured by the CCD only if they pass through a focusing device (e.g., an optical lens or optical fiber) and are focused upon a CCD element. However, the emitted photons may escape equally in all directions. In order to maximize their subsequent “capture” and quantification when utilizing a planar array (e.g., a DNA chip), the photons may be collected as close as possible to the point at which they are generated, e.g. immediately at the planar solid support. This may be accomplished by either: (i) utilizing optical immersion oil between the cover slip and a traditional optical lens or optical fiber bundle or, (ii) incorporating optical fibers directly into the cover slip itself. Similarly, when a thin, optically transparent planar surface is used, the optical fiber bundle may also be placed against its back surface, eliminating the need to “image” through the depth of the entire reaction/perfusion chamber.

The reaction event, e.g., photons generated by luciferase, may be detected and quantified using a variety of detection apparatuses, e.g., a photomultiplier tube, a CCD, CMOS, absorbance photometer, a luminometer, charge injection device (CID), or other solid state detector, as well as the apparatuses described herein. The quantification of the emitted photons may be accomplished by the use of a CCD camera fitted with a fused fiber optic bundle. The quantification of the emitted photons may also accomplished by the use of a CCD camera fitted with a microchannel plate intensifier. A back-thinned CCD may be used to increase sensitivity. CCD detectors are described in, e.g., Bronks, et al., 1995. Anal. Chem. 65: 2750-2757.

The CCD system may be a Spectral Instruments, Inc. (Tucson, Ariz.) Series 600 4-port camera with a Lockheed-Martin LM485 CCD chip and a 1-1 fiber optic connector (bundle) with 6-8 μm individual fiber diameters. This system may have 4096×4096, or greater than 16 million pixels and has a quantum efficiency ranging from 10% to >40%. Thus, depending on wavelength, as much as 40% of the photons imaged onto the CCD sensor may be converted to detectable electrons.

A fluorescent moiety may be used as a label and the detection of a reaction event may be carried out using a confocal scanning microscope to scan the surface of an array with a laser or other techniques such as scanning near-field optical microscopy (SNOM) are available which are capable of smaller optical resolution, thereby allowing the use of “more dense” arrays. For example, using SNOM, individual polynucleotides may be distinguished when separated by a distance of less than 100 nm, e.g., 10 nm×10 nm. Additionally, scanning tunneling microscopy (Binning et al., Helvetica Physica Acta, 55:726-735, 1982) and atomic force microscopy (Hanswa et al., Annu Rev Biophys Biomol Struct, 23:115-139, 1994) may be used.

The present invention has multiple aspects, illustrated by the following non-limiting examples.

### EXAMPLES

### Example 1

### Using λdoc Particles To Clone DNA Into A PAC Cloning Vector

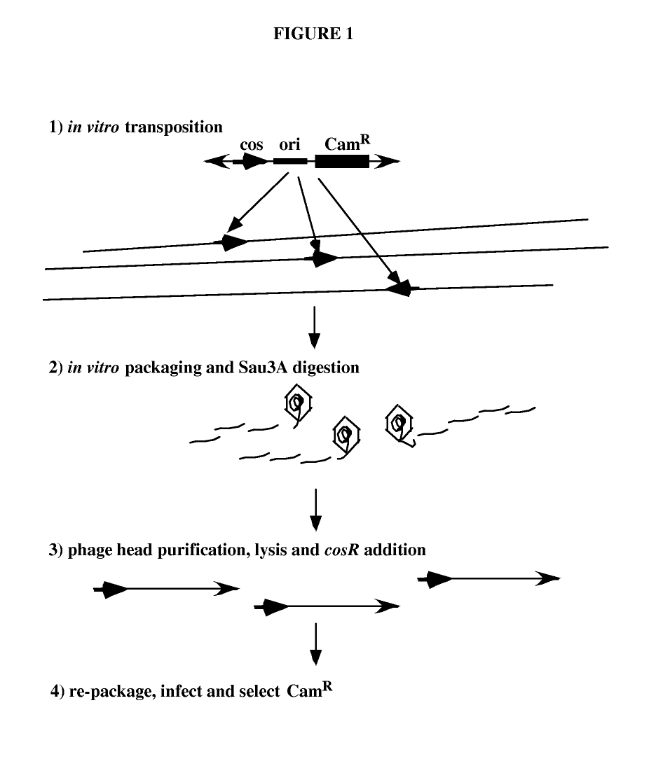

Using the Tn7 donor plasmid pGPS3 (New England Biolabs), a transposable cassette is constructed containing a λ cos site plus a complete copy of the pPAC\oriV vector (FIG. 1-1). The transposable cassette is then transposed into target DNA (FIG. 1-1). Transposition may be confirmed by Southern blotting.

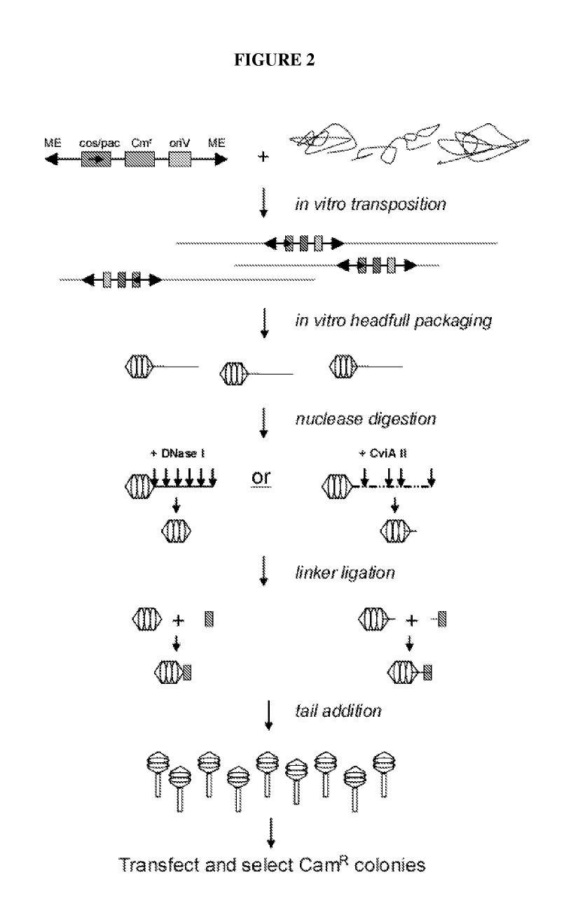

After transposition of the transposable element into target DNA it is packaged in vitro with λ extracts (FIG. 1-2). Proheads will fill but packaging may not be completed due to the lack of a second cos site. To provide the missing cos site, the preparation is digested with Sau3A to remove any DNA protruding from the heads. Next, phage tails are added to produce AdocL virions containing a headful of DNA. One end of the virion DNA terminates with cosL and the other with Sau3A overhangs.

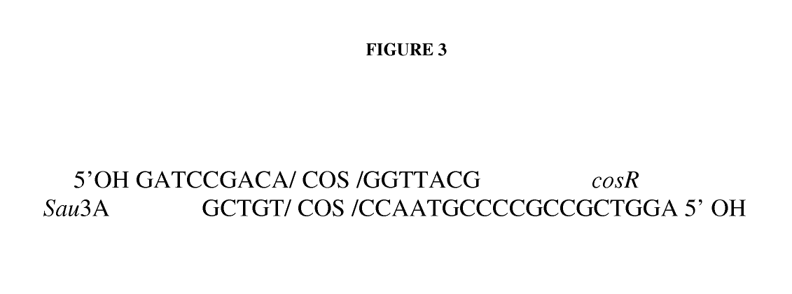

To circularize the packaged DNA molecules and form stable plasmids, a second cos site is added to the Sau3A by annealing a linker as shown in FIG. 3. The cos site added by the linker allows the DNA to circularize or form concatenates. Then a second round of in vitro packaging produces DNA molecules with cos overhangs on each end. The DNA molecules are circularized and repackaged in fully infectious form with the encapsidated DNA having λ cos sites at both ends. These preparations are then be used to introduce the PACs into cells at high efficiency and conveniently.

### Example 2

Additional Use of λdoc Particles To Clone DNA Into A PAC Cloning Vector

A cloning system was designed that used bacteriophage λ in vitro packaging for phage-based size selection and random cloning coinciding with a vector construct comprising an inducible origin of replication. This cloning system may be extended to incorporate bacteriophage P1 or P7 in vitro packaging as well as other larger (headful capacity) bacteriophages. Such a methodology for using bacteriophage in vitro packaging is shown in FIG. 2.

A phage packaging initiation recognition site is introduced into the target DNA by in vitro transposition. The transposon includes the phage-specific packaging initiation site, 19 by Tn5 mosaic ends for transposition, oriV for amplification of the cloned DNA, and a gene conferring antibiotic resistance for plasmid selection. Following transposition, a bacteriophage in vitro packaging system is used to package the cloned DNA. Packaging is initiated at the phage specific packaging site, and continuing until the headful capacity of the phage capsid is reached. This capacity, and therefore the clone insert size, may be based on the specific phage used.

The DNA protruding from the phage head and any unpackaged DNA is digested by DNase I or a 4-base recognition restriction endonuclease. The packaged DNA is protected from nuclease digestion. DNA linkers that allow later circularization of the clone are then ligated to the terminal end of the packaged DNA. Alternatively, the packaged DNA may be extracted from the phage heads, followed by linker ligation and repackaging. The phage tails are then added and the virions containing the cloned DNA are used to transfect E. coli followed by selection for antibiotic resistance.

### Example 3

Alternative Use of λdoc Particles To Clone DNA Into A PAC Cloning Vector

The procedure of Example 1 is performed up to the point that the proheads are filled with DNA and then cut with Sau3A. Instead of directly adding the phage tails, a cosR site is ligated to the λdoc particle while the DNA is protruding from the capsid using the linker of FIG. 3. After ligating the linkers, the tails are added as in Example 1. This simplifies the overall process because phage particles with normal cosR and cosL ends are used directly to introduce PACs into cells since it is not necessary to break open the capsids, circularize the DNA and repackage.

### Example 4

### In Vivo Transposon-Recombination Cloning

In Example 1 and 3, a significant amount of the cloning capacity is taken up by the vector. This is caused by the fact that the PAC vector has considerable length and thus occupies space in the capsid that otherwise could be devoted to cloned DNA. To address this limitation, a transposing cassette is prepared comprising modified Tn5 transposable ends flanking a cos site, a attP site for recombination, and Kan^{R} as a selectable marker. The transposon is randomly introduced in vitro into the genomic DNA, packaged from the cos site, Sau3A digested, the cosR linker added and the resultant fragments purified and re-packaged. Inserts are then introduced into a strain harboring a modified PAC vector containing a attP site, which is the cognate recombination site for attB. The strain expresses the Int protein from the pHS3-1 plasmid under the IPTG inducible P_{tac} promoter (Lee et al. 1990). The host is also IS⁻ to ensure that recombination only occurs with the plasmid. Once in the cell, the inserts cyclize via the cos overhangs then are recombined into the PAC through Int-mediated recombination. Recombinants are selected by plating on media containing antibiotics for both the vector (Cam^{R}) and the insert (Kan^{R}).

### Example 5

### Transposable Cloning Vectors

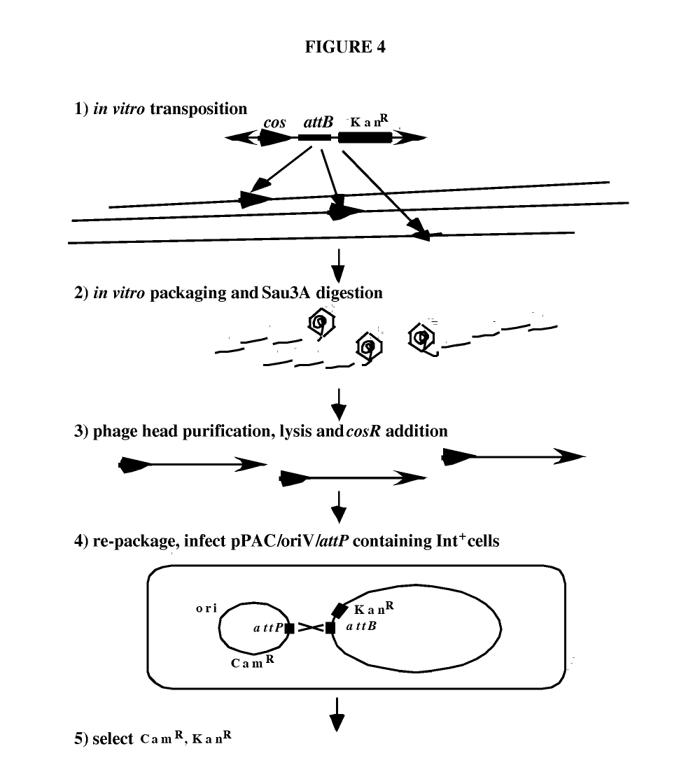

A series of transposable cloning vectors were constructed to include features for cloning using bacteriophage in vitro packaging. These features include: 1) a phage-specific packaging initiation sites for bacteriophage λ (cos) and a 162-bp P1 packaging initiation site (pac); 2) the inducible origin of replication, oriV; and 3) a kanamycin resistance gene flanked by the 19-bp mosaic end sequences for transposition. The mosaic ends in this construct are based on the hyperactive Tn5 in vitro transposition system and are designed to allow the creation of a transposon comprising the packaging sites, CAT (chloramphenicol acetyltransferase) gene, oriV, and the mosaic ends after digestion with the PvuII restriction enzyme. Integration of the transposon into target DNA can be confirmed by selection on chloramphenicol (Cam^{r}) and further screened for sensitivity to kanamycin (Kan^{s}). The locations of the cos and pac phage packaging initiation sites are designed so that following integration, packaging will begin at these sites, continue clockwise through the vector (see FIG. 4), and ultimately into the adjacent target DNA. This allows the clone to be stably selected and maintained as a plasmid following packaging, transfection, and circularization.

A series of three plasmids were constructed, containing cos, pac, and both cos and pac. Finally, the 19-bp mosaic ends sequences flanking the Kan^{r} gene were added to the three vectors for the final products. Each component of the vector series has been individually tested and shown to function as expected. All three vectors were shown to be efficiently transposed in vivo and integrated into a DH10B genome. Approximately 98% of transformants analyzed following transposition were Cam^{r}. Kan^{s} confirming integration. Southern blotting of genomic DNA from the candidates confirmed random integration into the genome.

cos functionality was confirmed by two experiments. A simple digestion with purified λ terminase (Epicentre) confirmed cos cleavage of the vectors. Also, an in vivo cosmid packaging assay was performed to determine if concatemers composed of plasmid multimers were able to be packaged by a λ prophage in vivo. Concatemers produced from the two vectors containing cos were efficiently packaged as measured by the number of Cam^{r} transducing particles, while the vector with pac alone was unable to be packaged by λ in vivo.

### Example 6

### Ligation of Phage Packaging Site

A packaging site is introduced into target DNA by ligating a linearized transposable cloning vector directly to partially digested genomic DNA. An EcoRI, BamHI, and HindIII site (or an entire multiple cloning site) is introduced into the transposable cloning vector at a unique PmeI site situated between the tL3 terminator and mosaic end. If an EcoRI partial digest is used, the transposable cloning vector is digested by EcoRI and PvuII and purified, resulting in a linearized transposable cloning vector with the blunt ended PvuII and pac/cos site at one end and an EcoRI sticky end at the opposite end. The EcoRI/PvuII double digest leads to unidirectional packaging with clones containing the vector elements.

The linearized transposable cloning vector is ligated to partially digested genomic DNA. An in vitro packaging reaction is then performed, initiating packaging at the packaging site on the transposable cloning vector, through the components of the transposable cloning vector, and continuing into the ligated genomic DNA until the headful capacity of the phage head is reached. Similar to introduction of the packaging initiation site by transposition, the protruding DNA is digested, appropriate linkers ligated for circularization of the DNA, phage tails added, and the virion containing the cloned DNA used to transfect E. coli.

### Example 7

### In Vitro Packaging with λ or P1

Commercially available λ in vitro packaging extracts combine the stage 1 and stage 2 packaging extracts as a single-tube system. The cloning system may sequentially use the two packaging stages sequentially. Extracts from the two traditional complementary lysogenic E. coli strains BHB2690 (stage 1) and BHB2688 (stage 2) were generated and tested. The efficiency of the two-stage packaging system was comparable to that of commercially available single-tube systems. In addition, we were able to demonstrate cos cleavage of the packaging vectors using the stage 1 extract, confirming the functionality of the vectors described in Example 5. For an in vitro packaging system for bacteriophage P1, we generated stage 1 and stage 2 extracts of P1 lysogens from strains NS3208 and NS3210, respectively (Coren et al., J Mol Biol, 249:176-84, 1995). We also demonstrated cleavage of the pac site of the packaging vectors using the stage 1 (pacase) extract.

### Example 8

Cloning Genomic DNA Using λ Phage Packaging and Affinity Purification of Phage Capsids

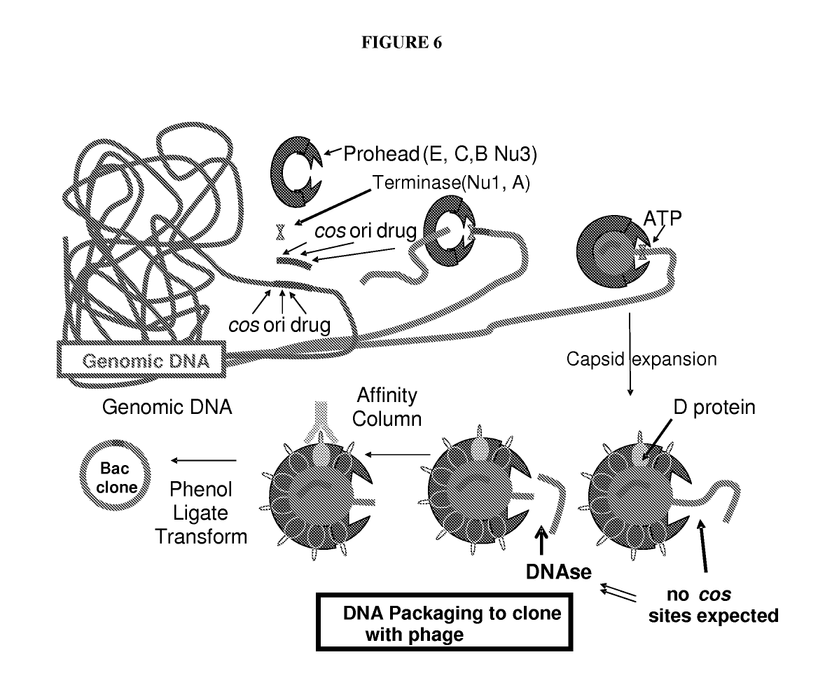

This example describes cloning of DNA using λ phage packaging and affinity purification of phage cap sids. A nucleic acid containing a cos site, an origin of replication, and a drug resistance marker are randomly inserted into genomic DNA, for example by in vitro transposition. The λ terminase (containing products of the Nul and A genes) binds to the cos site and to the λ prohead, and then cuts the DNA at the cos site (FIG. 6). Then, an ATP-driven motor stuffs the DNA into the prohead. The viral capsid expands as DNA is inserted into it by the addition of D protein to the capsid. This process continues until about 50 kb of DNA fills the capsid. DNA that remains hanging out of the capsid can be cut with a frequently-cutting restriction site. Only DNA that is outside the capsid can be cut by the restriction enzyme because DNA that is inside the capsid is protected.

Following restriction of the DNA, the phage capsid is affinity purified using anti-D protein antibody bound to a column. DNA from purified capsids can be isolated by phenol extraction. The DNA is then circularized and transformed into bacteria.

### Example 9

### Cloning Genomic DNA Using P22HT Phage Packaging

This example describes cloning of genomic DNA using P22HT phage packaging. P22HT terminase, containing the Gp2 and Gp3 proteins, binds to genomic DNA at random sites and cuts it (FIG. 7). The terminase remains bound to the DNA and stuffs it into a P22 prohead until the prohead is full. As the capsid expands, it stretches and undergoes a conformational change that expels the terminase, still attached to the broken DNA. The terminase can then insert the DNA into a new prohead, beginning a new cycle of DNA packaging. The terminase can continue many cycles of successive packaging of adjacent genomic DNA segments.

To clone the packaged DNA, phage capsids can be affinity purified using a column containing antibodies that are capable of binding an epitope that is present on the outside of the expanded capsid. Capsids can also be isolated by differential sedimentation or isopycnic centrifugation. These methods can be accompanied by DNase digestion. The DNA can be cloned by further extracting, circularizing, and transforming it into bacteria as described above.

### Example 10

### Characterizing the Sequences of the Ends of Genomic DNA Cloned by Phage Packaging

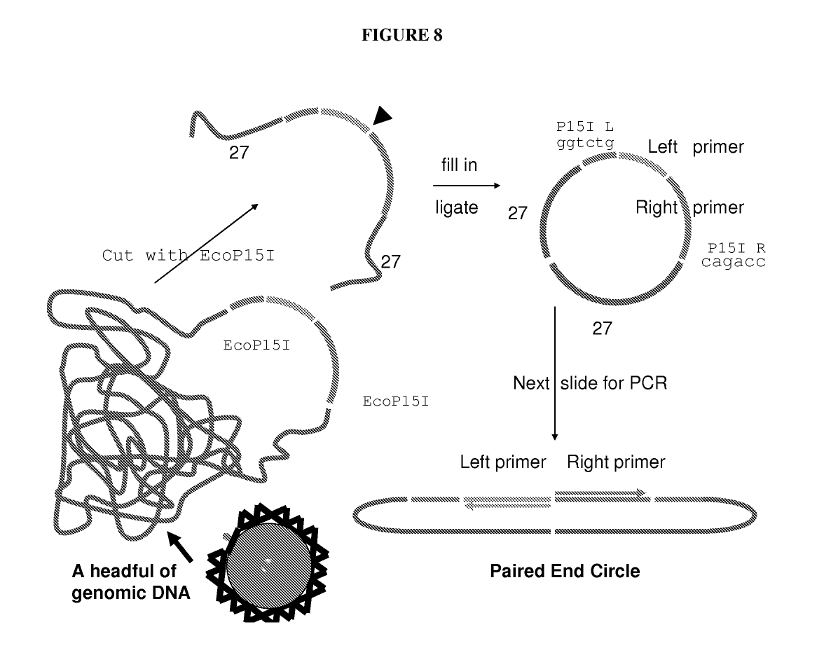

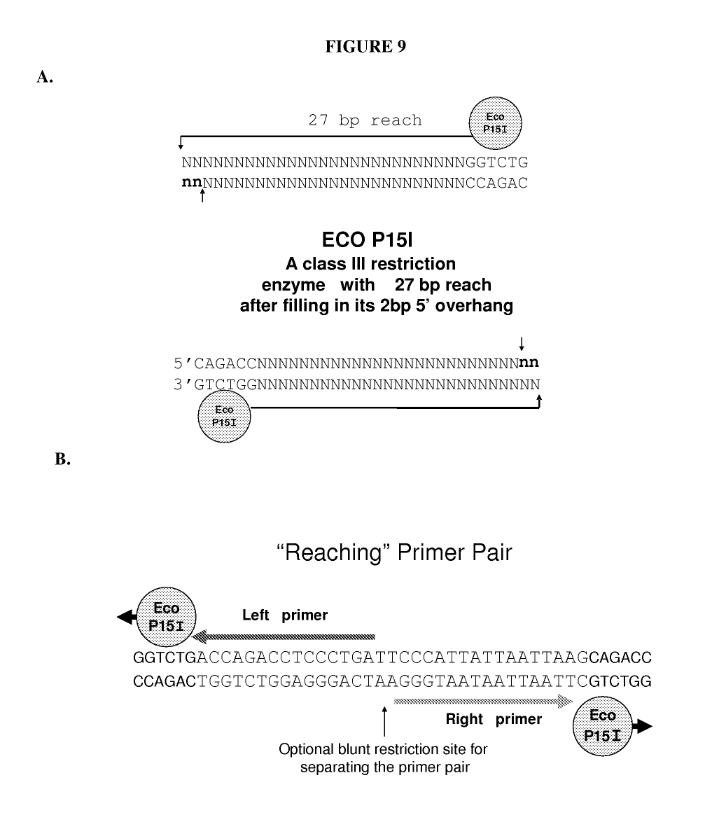

This example describes how the sequences of the ends of genomic DNA that has been packaged using a phage-based system can be characterized. The genomic DNA is first isolated from phage capsids. Next, a nucleic acid containing two outward-oriented reaching primer sequences flanked by EcoP15I sites is ligated to the ends of the isolated DNA fragment (FIG. 8). FIG. 9B shows the structure of the reaching primer pair nucleic acid. The reaching primer pair fragment is ligated to the isolated DNA at a dilution that is low enough to avoid ligating the reaching primer pair fragment to two different genomic DNA molecules. The ends of the reaching primer pair fragment may not be phosphorylated to avoid forming monomer circles with it.

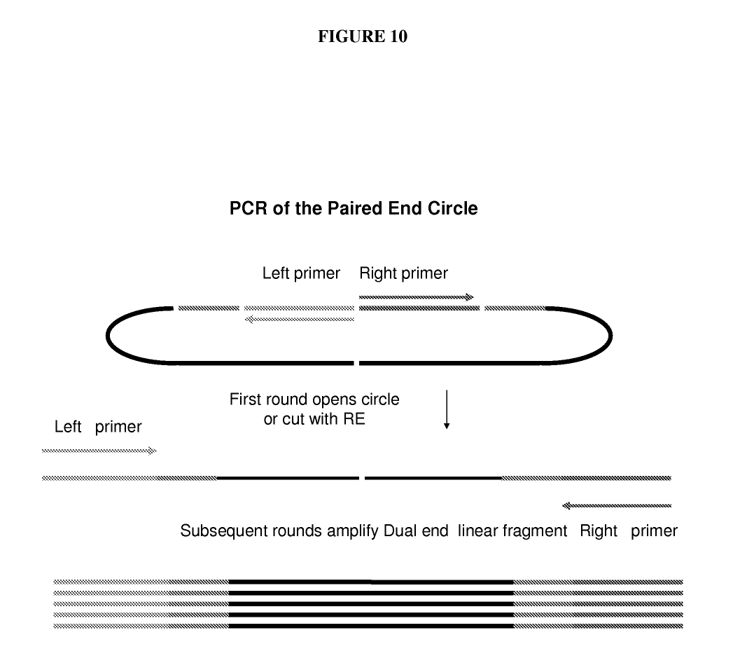

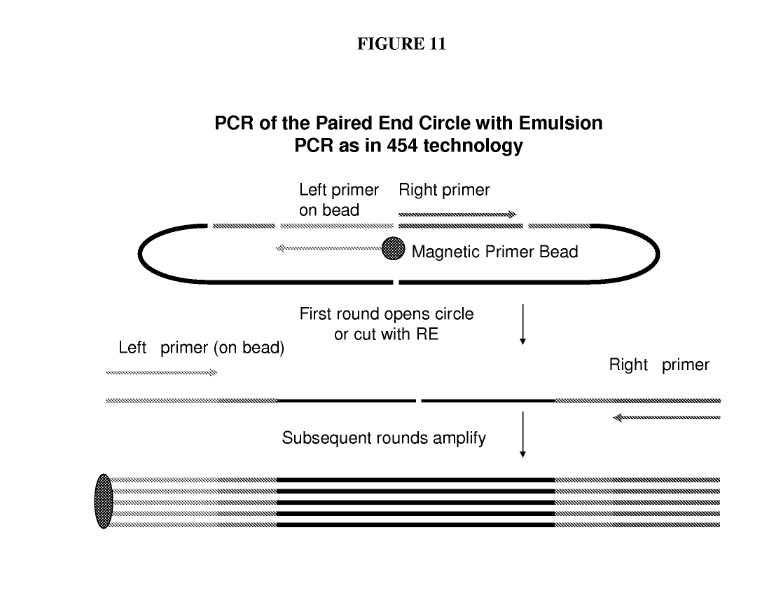

Following ligation, the DNA is digested with EcoP15I, which cuts DNA 27 by away from the EcoP15I recognition site. EcoRDigestion with this restriction enzyme releases a DNA fragment that contains the two reaching primer sequences, the flanking EcoP15I sites, and the 27 by of genomic DNA at each end. The genomic DNA in the release fragment represents each end of the packaged genomic DNA. The released fragment can be filled-in. FIG. 9A shows how the EcoP151-cut ends of the released fragment can be filled-in to create blunt ends. The released blunted fragment can then be ligated to create a paired end circle DNA. This DNA can then be PCR amplified using the two reaching primers (FIG. 10), and the amplified product may then be sequenced. If there is a restriction site between the reaching primer pair sites, the paired end circle may be linearized by cutting with the appropriate restriction enzyme before PCR amplification. The DNA may also be PCR amplified and directly sequenced by performing the amplification using solid phase nucleic acid amplification. The approach described above can also be adapted with one of the reaching primers bound to a magnetic bead for use in bead emulsion PCR amplification and sequencing (FIG. 11).

1.-76. (canceled)

77. A method of cloning a nucleic acid from a target nucleic acid comprising:
(a) contacting a target nucleic acid with a P22HT terminase;
(b) initiating packaging, whereby the nucleic acid to be cloned from the target nucleic acid is packaged; and
(c) isolating the packaged nucleic acid.

78. The method of claim 77, wherein the target nucleic acid is an entire genome.

79. The method of claim 77 wherein the packaged nucleic acid is packaged in a capsid.

80. The method of claim 79 wherein packaging occurs in vitro.

81. The method of claim 79 wherein packaging occurs in vivo.

82. The method of claim 77, wherein the target nucleic acid is chromosomal DNA.

83. The method of claim 77, wherein the target nucleic acid is a vector.

84. The method of claim 77, wherein the P22HT terminase is a mutant Gp3 protein.

85. The method of claim 77, wherein the P22HT terminase comprises a HT105/1 mutation.

86. The method of claim 77, wherein the P22HT terminase recognizes DNA with lower specificity as compared to a wild-type P22HT terminase.

87. The method of claim 86, wherein the P22HT terminase binds to and cleaves DNA at random locations.
